(19) United States
(12) Patent Application Publication
(10) Pub. No.: US 2010/0286480 A1
(43) Pub. Date: Nov. 11, 2010

(54) Surgical instrument

(76) Inventors: William J. Peine, Ashland, MA (US); Woojin Lee, Hopkinton, MA (US); Andres Chamorro, Waltham, MA (US)

Correspondence Address: David M. Driscoll, Esq., 1201 Canton Avenue, Milton, MA, 02186, US

(21) Appl. No.: 12/387,716

(22) Filed: May 6, 2009

(51) Int. Cl. A61B 1/045; A61B 1/00

(52) U.S. Cl. 600/131; 600/139

(57) ABSTRACT

A surgical instrument that includes an instrument shaft having proximal and distal ends, a tool disposed from the distal end of the instrument shaft, a control handle disposed from the proximal end of the instrument shaft, a distal motion member for coupling the distal end of the instrument shaft to the tool, a proximal motion member for coupling the proximal end of the instrument shaft to the handle, actuation means extending between the distal and proximal motion members for coupling motion of the proximal motion member to the distal motion member for controlling the positioning of the tool and a locking mechanism for fixing the position of the tool at a selected position and having locked and unlocked states.

### TECHNICAL FIELD

The present invention relates in general to medical instruments, and more particularly to manually-operated surgical instruments that are intended for use in minimally invasive surgery or other forms of surgical or medical procedures or techniques. The instrument described herein is primarily for a laparoscopic procedure, however, it is to be understood that the instrument of the present invention can be used for a wide variety of other procedures, including intraluminal procedures.

### BACKGROUND OF THE INVENTION

Endoscopic and laparoscopic instruments currently available in the market are extremely difficult to learn to operate and use, mainly due to a lack of dexterity in their use. For instance, when using a typical laparoscopic instrument during surgery, the orientation of the tool of the instrument is solely dictated by the location of the target and the incision. These instruments generally function with a fulcrum effect using the patients own incision area as the fulcrum. As a result, common tasks such as suturing, knotting and fine dissection have become challenging to master. Various laparoscopic instruments have been developed over the years to overcome this deficiency, usually by providing an extra articulation often controlled by a separately disposed control member for added control. However, these instruments still do not provide enough dexterity to allow the surgeon to perform common tasks such as suturing, particularly at any arbitrarily selected orientation. Also, existing instruments of this type do not provide an effective way to hold the instrument in a particular position. Moreover, existing instruments require the use of both hands in order to effectively control the instrument.

Other improvements in surgical instruments are disclosed in the following U.S. patents commonly owned with the present assignee. They are U.S. Pat. Nos. 7,147,650; 7,338,513 and 7,364,582. These patents show various instrument constructions, as well as locking mechanisms, including means for pinching cabling to hold a position. Reference is also made to a co-pending application commonly owned with the present invention. That is application Ser. No. 11/649,352 filed on Jan. 2, 2007. This application discloses other forms of locking means including a ball and socket arrangement and associated cinch ring.

An object of the present invention is to provide an improved laparoscopic or endoscopic surgical instrument that allows the surgeon to manipulate the tool end of the surgical instrument with greater dexterity.

Another object of the present invention is to provide an improved surgical or medical instrument that has a wide variety of applications, through incisions, through natural body orifices or intraluminally.

A further object of the present invention is to provide an improved medical instrument that is characterized by the ability to lock the instrument in a pre-selected particular position.

Another object of the present invention is to provide a locking feature that is an important adjunct to the other controls of the instrument enabling the surgeon to lock the instrument once in the desired position. This makes it easier for the surgeon to thereafter perform surgical procedures without having to, at the same time, hold the instrument in a particular bent configuration.

Still another object of the present invention is to provide an improved medical instrument that can be effectively controlled with primarily only a single hand of the user.

Another object of the present invention is to provide a medical instrument in which the associated locking mechanism can be made more compact.

Still another object of the present invention is to provide a medical instrument in which there is greater flexibility as to the location of the means for carrying out the locking feature.

### SUMMARY OF THE INVENTION

To accomplish the foregoing and other objects, features and advantages of the present invention there is provided a surgical instrument that includes an instrument shaft having proximal and distal ends; a tool disposed from the distal end of the instrument shaft; a control handle coupled from the proximal end of the instrument shaft; a distal motion member for coupling the distal end of said instrument shaft to said tool; a proximal motion member for coupling the proximal end of said instrument shaft to said handle; actuation means extending between said distal and proximal motion members for coupling motion of said proximal motion member to said distal motion member for controlling the positioning of said tool; and a locking mechanism for fixing the position of the tool at a selected position and having locked and unlocked states. The locking mechanism including one of a cable array and rod array disposed about said proximal motion member and a locking ring disposed about said proximal motion member and having locked and released positions, and in said locked position engaging said one of a cable array and rod array.

In accordance with other aspects of the present invention the surgical instrument may further include a rotation means disposed adjacent the control handle and rotatable relative to the control handle for causing a corresponding rotation of the instrument shaft and tool; at least the proximal motion member may comprise a proximal bendable member, with the rotation means comprising a rotation knob that is adapted to rotate the tool about a distal tool roll axis and being disposed between the control handle and proximal bendable member; the control handle may comprise a pistol grip handle having an engagement horn to assist in holding the handle; the rotation means may comprises a rotation knob that is disposed at the distal end of the handle and the horn is disposed proximally of the rotation knob and on the top of the pistol grip handle; including an actuation lever supported from the pistol grip handle at a pivot point at the proximal end of the handle; the actuation lever may have a free end with a finger loop for receiving a users finger to control the lever; and preferably including a tool actuation cable that extends from the tool to the handle.

In accordance with still other aspects of the present invention the surgical instrument may further have the cable sections extend about an outer circumferential surface of the handle, the locking ring has an internal cam that pinches the cable section against a rib on the handle hub and including a spring means in each cable section; the one of a cable array and rod array may comprise a cable array including a plurality of cable sections that extend about the proximal motion member, and a plurality of pulleys mounted in a handle hub and supporting respective cable sections; the handle hub may have peripheral slots, said locking ring has peripherally disposed internal cams that pinch the respective cable sections against a pulley and further including spring means in at least one cable section; the one of a cable array and rod array may comprise a rod array including a plurality of separate rods that extend about the proximal motion member, and a plurality of housings that are supported by a handle hub and include a corresponding plurality of split balls that receive respective rods; there may be provided a plurality of peripherally disposed internal cams on the locking ring for engaging the split balls to lock the position; the locking ring, proximal motion member and instrument shaft may be removable from the control handle, and include a quick disconnect means for releasably engaging a tool actuation cable means of the instrument; the one of a cable array and rod array may comprise a cable array including a plurality of cable sections that extend about the proximal motion member, a plurality of sheaves mounted in a handle hub and supporting respective cable sections, a fixed position anchor disc disposed about the instrument shaft for securing one end of each cable section, a fixed anchor for securing an opposite end of each cable section and a spring disposed in each cable section; and further including capstan means for supporting at least some of the cable sections.

In accordance with another version of the instrument of the present invention there is provided a medical instrument having a proximal control handle and a distal tool that are intercoupled by an elongated instrument shaft that is meant to pass internally of an anatomic body, proximal and distal bendable members that respectively intercouple said proximal control handle and said distal tool with said instrument shaft, cable actuation means disposed between said bendable members, said control handle having proximal and distal ends, an actuation lever for controlling said distal tool, means for pivotally supporting said actuation lever from the proximal end of said handle, and a locking mechanism for fixing the position of the tool at a selected position and having locked and unlocked states, said locking mechanism including one of a cable array and rod array disposed about said proximal bendable member and a locking ring disposed about said proximal bendable member and having locked and released positions, and in said locked position engaging said one of a cable array and rod array.

In accordance with still another aspect of the present invention, the angle locking means need not be in the form of a locking ring, but may include a locking mechanism that is supported at the handle. The use of cabling in particular lends itself well to being able to relocate the locking mechanism to any one of a number of different positions on the instrument. That makes it more comfortable in the use of the instrument. Even the embodiment that uses pulleys or the like can have the locking mechanism easily relocated to the handle area of the instrument.

### BRIEF DESCRIPTION OF THE DRAWINGS

It should be understood that the drawings are provided for the purpose of illustration only and are not intended to define the limits of the disclosure. The foregoing and other objects and advantages of the embodiments described herein will become apparent with reference to the following detailed description when taken in conjunction with the accompanying drawings in which:

FIG. 1 is a perspective view of a first embodiment of the present invention;

FIG. 2 is a fragmentary cross-sectional side view of the instrument of FIG. 1;

FIG. 3 is a cross-sectional view similar to FIG. 2 but showing the instrument shaft in an angled position;

FIG. 4 is a cross-sectional view taken along line 4-4 of FIG. 2;

FIG. 5 is a cross-sectional view taken along line 5-5 of FIG. 3;

FIG. 6 is a fragmentary perspective view of the embodiment of FIG. 1 with the locking ring removed for simplicity;

FIG. 6A is a schematic perspective view of the cabling mechanism of FIG. 6;

FIG. 6B is a schematic perspective view similar to that shown in FIG. 6A but with the instrument rotated 35 degrees in the “X” direction;

FIG. 7 is a fragmentary cross-sectional side view of a second embodiment of the present invention;

FIG. 8 is a cross-sectional view similar to that shown in FIG. 7 but illustrating the instrument shaft in an angled position;

FIG. 9 is a cross-sectional view taken along line 9-9 of FIG. 7;

FIG. 10 is a cross-sectional view taken along line 10-10 of FIG. 8;

FIG. 11 is a fragmentary perspective view of the instrument of FIG. 7 with the locking ring removed for simplicity of description;

FIG. 11A is a schematic perspective view of the cabling mechanism of FIG. 11;

FIG. 11B is a schematic perspective view similar to that shown in FIG. 11A but with the instrument rotated 35 degrees in the “Y” direction;

FIG. 12 is a fragmentary cross-sectional side view of a third embodiment of the present invention;

FIG. 13 is a cross-sectional side view similar to that shown FIG. 12 but with the instrument shaft in an angled position.

FIG. 14 is a cross-sectional view taken along line 14-14 of FIG. 12;

FIG. 15 is a cross-sectional view taken along line 15-15 of FIG. 13;

FIG. 16 is a cross-sectional view taken along line 16-16 of FIG. 13;

FIG. 17 is a fragmentary enlarged detail perspective view of a locking cam arrangement for the embodiment of FIG. 12;

FIG. 18 is a fragmentary perspective view of the embodiment of FIG. 12 with the locking ring removed for simplicity of description;

FIG. 19 is an exploded perspective view of the embodiment shown in FIG. 12;

FIG. 20 is a fragmentary cross-sectional side view of a fourth embodiment of the present invention;

FIG. 21 is a cross-sectional side view similar to that shown FIG. 20 but with the instrument shaft and proximal bendable member removed from the handle;

FIG. 22 is an exploded perspective view of the embodiment of FIG. 20;

FIG. 22A is a fragmentary cross-sectional view taken along line 22A-22A of FIG. 22;

FIG. 23 is a perspective view of a fifth embodiment of the present invention;

FIG. 23A is a schematic perspective view of the cabling mechanism of FIG. 23;

FIG. 23B is a schematic perspective view similar to that shown in FIG. 23A but with the instrument rotated in both the “X” and “Y” directions at the same time;

FIG. 24 is a cross-sectional view taken along line 24-24 of FIG. 23;

FIG. 25 is a cross-sectional view taken along line 25-25 of FIG. 23;

FIG. 26 is a schematic cross-sectional view similar to FIG. 24 but showing a cable scheme for a sixth embodiment of the present invention;

FIG. 27 is a schematic cross-sectional view similar to FIG. 24 but showing a cable scheme for a seventh embodiment of the present invention;

FIG. 28 is a fragmentary perspective view of an embodiment of the present invention in which the angle locking member is disposed at the handle, particularly at the horn thereof;

FIG. 29 is a fragmentary perspective view like that shown in FIG. 28 with the bellows removed so that the cabling can be seen;

FIG. 29A is a schematic perspective view of the cabling mechanism of FIG. 29;

FIG. 29B is a schematic perspective view similar to that shown in FIG. 29A but with the instrument rotated in both the “X” and “Y” directions at the same time;

FIG. 30 is a cross-sectional side view of the instrument in FIGS. 28 and 29;

FIG. 30A is a cross-sectional view taken along line 30A-30A of FIG. 30;

FIG. 31 is a cross-sectional side view of the instrument in FIGS. 28 and 29 with the instrument in a bent condition; and

FIGS. 31A and 31B are fragmentary cross-sectional views taken at the slide button for the respective released and locked positions thereof.

### DETAILED DESCRIPTION OF THE PREFERRED EMBODIMENT

The instrument of the present invention may be used to perform minimally invasive procedures. “Minimally invasive procedure,” refers herein to a surgical procedure in which a surgeon operates through a small cut or incision, the small incision being used to access the operative site. In one embodiment, the incision length ranges from 1 mm to 20 mm in diameter, preferably from 5 mm to 10 mm in diameter. This procedure contrasts those procedures requiring a large cut to access the operative site. Thus, the flexible instrument is preferably used for insertion through such small incisions and/or through a natural body lumen or cavity, so as to locate the instrument at an internal target site for a particular surgical or medical procedure. The introduction of the surgical instrument into the anatomy may also be by percutaneous or surgical access to a lumen, vessel or cavity, or by introduction through a natural orifice in the anatomy.

In addition to use in a laparoscopic procedure, the instrument of the present invention may be used in a variety of other medical or surgical procedures including, but not limited to, colonoscopic, upper GI, arthroscopic, sinus, thorasic, prostate, transvaginal, orthopedic and cardiac procedures. Depending upon the particular procedure, the instrument shaft may be rigid, semi-rigid or flexible.

Although reference is made herein to a “surgical instrument,” it is contemplated that the principles of this invention also apply to other medical instruments, not necessarily for surgery, and including, but not limited to, such other implements as catheters, as well as diagnostic and therapeutic instruments and implements.

There are several different embodiments that are described herein. Basically, in all these embodiments preferably both the tool and handle motion members or bendable members are capable of bending in any direction. They are interconnected via cables in such a way that a bending action at the proximal member provides a related bending at the distal member. The proximal bending is controlled by a motion or deflection of the control handle by a user of the instrument. In other words the surgeon grasps the handle and once the instrument is in position any motion at the handle (deflection) immediately controls the proximal bendable member which, in turn, via cabling controls a corresponding bending or deflection at the distal bendable member.

In this description reference is made to bendable members. These members may also be referred to as turnable members or flexible members. In the descriptions set out herein, terms such as “bendable section,” “bendable segment,” “bendable motion member,” or “turnable member” refer to an element of the instrument that is controllably bendable in comparison to an element that is pivoted at a joint. The term “movable member” is considered as generic to bendable sections and joints. The bendable elements of the present invention enable the fabrication of an instrument that can bend in any direction without any singularity and that is further characterized by a ready capability to bend in any direction. One form of bendable members shown herein includes a single unitary or uni-body structure. Another form of bendable member disclosed herein is a ball and rider structure.

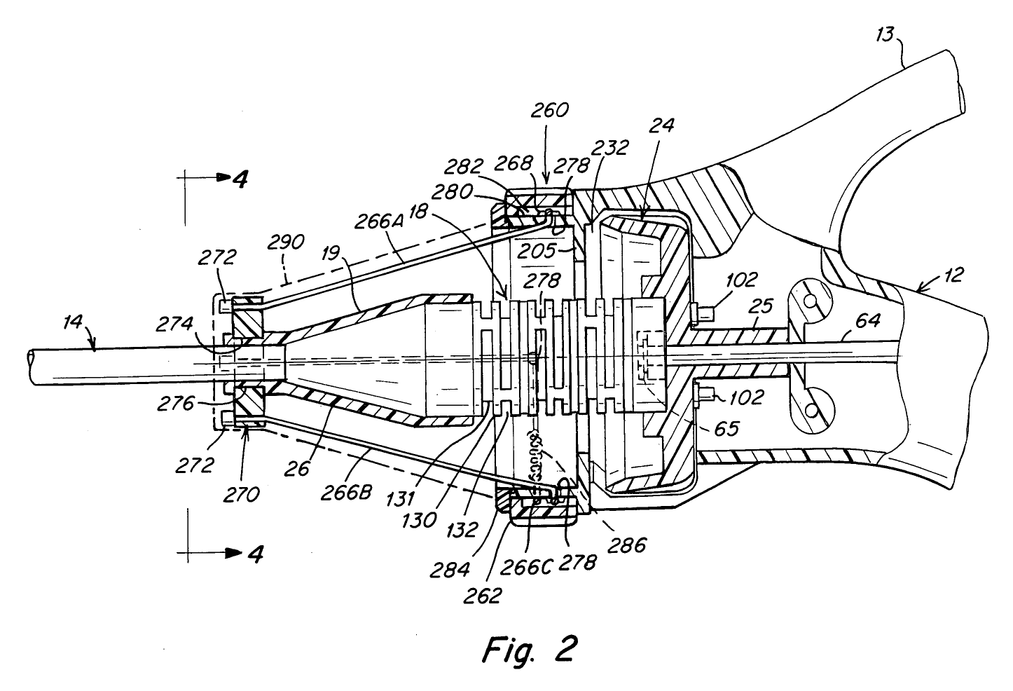

A definition of these bendable members is—an instrument element, formed either as a controlling means or a controlled means, and that is capable of being constrained by tension or compression forces to deviate from a straight line to a curved configuration without any sharp breaks or angularity—. Bendable members may be in the form of unitary structures, such as shown herein in FIG. 2, may be constructed of engageable discs, or the like, may include bellows arrangements or may comprise a movable ring assembly. For other forms of bendable members refer to co-pending applications Ser. No. 11/505,003 filed on Aug. 16, 2006 and Ser. No. 11/523,103 filed on Sep. 19, 2006, both of which are hereby incorporated by reference herein in their entirety. A definition of a “unitary” or “uni-body” structure is,—a structure that is constructed only of a single integral member and not one that is formed of multiple assembled or mated components—.

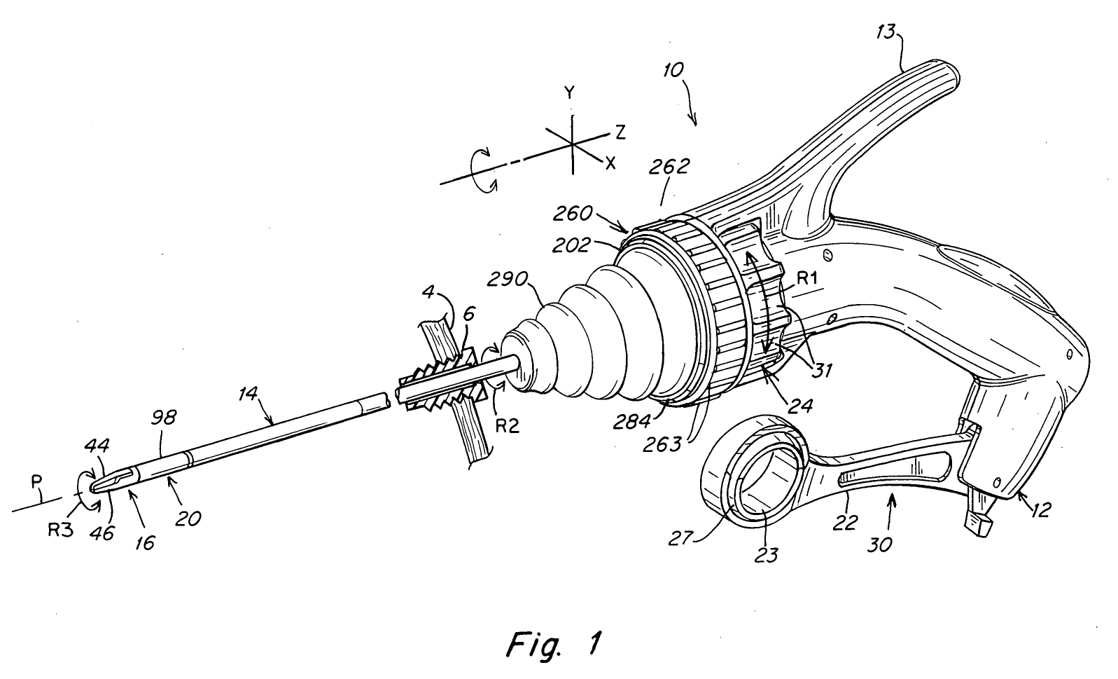

FIG. 1 is a perspective view of a first embodiment of the surgical instrument 10 of the present invention. In this surgical instrument both the tool and handle motion members or bendable members are capable of bending in any direction. They are interconnected via cables (preferably four cables) in such a way that a bending action at the proximal member provides a related bending at the distal member. The proximal bending is controlled by a motion or deflection of the control handle by a user of the instrument. In other words the surgeon grasps the handle and once the instrument is in position any motion (deflection) at the handle immediately controls the proximal bendable member which, in turn, via cabling controls a corresponding bending or deflection at the distal bendable member. This action, in turn, controls the positioning of the distal tool.

The proximal member is preferably generally larger than the distal member so as to provide enhanced ergonomic control. In the illustrated embodiment the ratio of proximal to distal bendable member diameters may be on the order of three to one. In one version in accordance with the invention there may be provided a bending action in which the distal bendable member bends in the same direction as the proximal bendable member. In an alternate embodiment the bendable, turnable or flexible members may be arranged to bend in opposite directions by rotating the actuation cables through 180 degrees, or could be controlled to bend in virtually any other direction depending upon the relationship between the distal and proximal support points for the cables.

As has been noted the, amount of bending motion produced at the distal bending member is determined by the dimension of the proximal bendable member in comparison to that of the distal bendable member. In the embodiment described the proximal bendable member is generally larger than the distal bendable member, and as a result, the magnitude of the motion produced at the distal bendable member is greater than the magnitude of the motion at the proximal bendable member. The proximal bendable member can be bent in any direction (about 360 degrees) controlling the distal bendable member to bend in either the same or an opposite direction, but in the same plane at the same time. Also, the surgeon is able to bend and roll the instrument's tool about its longitudinal axis to any orientation simply by rolling the axial rotation knob about rotation direction R1.

FIG. 1 shows a first embodiment of the instrument of the present invention. Further details are illustrated in FIGS. 1-6. FIG. 1 depicts the surgical instrument 10 in position, as may occur during a surgical procedure. For example, the instrument may be used for laparoscopic surgery through the abdominal wall, such as shown at 4 in FIG. 1. For this purpose there is provided an insertion site at which there is disposed a cannula or trocar 6. The shaft 14 of the instrument 10 is adapted to pass through the cannula or trocar 6 so as to dispose the distal end of the instrument at the operative site. The end effector 16 is depicted in FIG. 1 at such an operative site with the cannula or trocar 6 at the incision point in the skin 4. The embodiment of the instrument shown in FIG. 1 is typically used with a sheath 98 to keep bodily fluids from entering the distal bending member 20.

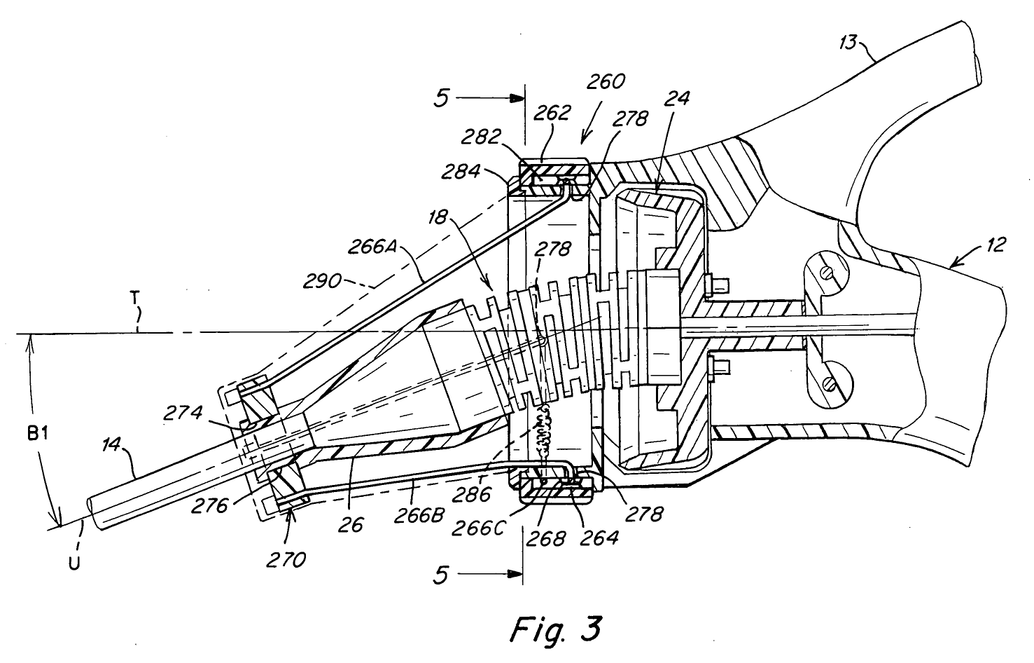

A rolling motion can be carried out with the instrument of the present invention. This can occur by virtue of the rotation of the rotation knob 24 relative to the handle 12 about axis T (refer to FIG. 3). This is represented in FIG. 1 by the rotation arrow R1. When the rotation knob 24 is rotated, in either direction, this causes a corresponding rotation of the instrument shaft 14. This is depicted in FIG. 1 by the rotational arrow R2. This same motion also causes a rotation of the distal bendable member and end effector 16 about an axis that corresponds to the instrument tip, depicted in FIG. 1 as about the longitudinal distal tip or tool axis P. A rolling motion can also be provided by rotation of the instrument handle about the instrument shaft axis.

Any rotation of the rotation knob 24 while the instrument is locked (or unlocked) maintains the instrument tip at the same angular position, but rotates the orientation of the tip (tool). For a further explanation of the tip rotational feature refer to co-pending application Ser. No. 11/302,654, filed on Dec. 14, 2005, particularly FIGS. 25-28, which is hereby incorporated by reference in its entirety.

The handle 12, via proximal bendable member 18, may be tilted at an angle to the instrument shaft longitudinal center axis. This tilting, deflecting or bending may be considered as in the plane of the paper. By means of the cabling this action causes a corresponding bend at the distal bendable member 20 to a position wherein the tip is directed along an axis and at a corresponding angle to the instrument shaft longitudinal center axis. The bending at the proximal bendable member 18 is controlled by the surgeon from the handle 12 by manipulating the handle in essentially any direction including in and out of the plane of the paper in FIG. 1. This manipulation directly controls the bending at the proximal bendable member. Refer to FIG. 3 in which there is shown the axis U corresponding to the instrument shaft longitudinal axis. Refer also to the proximal bend angle B1 between axes T and U which will control a corresponding distal bend angle between axes U and P.

Thus, the control at the handle is used to bend the instrument at the proximal motion member to, in turn, control the positioning of the distal motion member and tool. The “position” of the tool is determined primarily by this bending or motion action and may be considered as the coordinate location at the distal end of the distal motion member. Actually, one may consider a coordinate axis at both the proximal and distal motion members as well as at the instrument tip, as illustrated in FIG. 1 by the axes X, Y and Z. This positioning is in three dimensions. Of course, the instrument positioning is also controlled to a certain degree by the ability of the surgeon to pivot the instrument at the incision point (cannula 6) with the incision location being a fulcrum point. Position can also be controlled by rotation of the handle about the instrument shaft axis. The “orientation” of the tool, on the other hand, relates to the rotational positioning of the tool, from the proximal rotation control member, about the illustrated distal tip or tool axis P.

In the drawings a set of jaws 44, 46 is depicted, however, other tools or devices may be readily adapted for use with the instrument of the present invention. These include, but are not limited to, cameras, detectors, optics, scope, fluid delivery devices, syringes, etc. The tool may include a variety of articulated tools such as: jaws, scissors, graspers, needle holders, micro dissectors, staple appliers, tackers, suction irrigation tools and clip appliers. In addition, the tool may include a non-articulated tool such as: a cutting blade, probe, irrigator, catheter or suction orifice.

The surgical instrument of FIG. 1 shows a first embodiment of a surgical instrument 10 according to the invention in use and may be inserted through a cannula at an insertion site through a patient's skin. Many of the components shown herein, such as the instrument shaft 14, end effector 16, distal bending member 20, and proximal bending member 18 may be similar to and interact in the same manner as the instrument components described in the co-pending U.S. application Ser. No. 11/185,911 filed on Jul. 20, 2005 and hereby incorporated by reference herein in its entirety. Many other components shown herein, particularly at the handle end of the instrument may be similar to components described in the co-pending U.S. application Ser. No. 11/528,134 filed on Sep. 27, 2006 and hereby incorporated by reference herein in its entirety. Also incorporated by reference in their entirety are U.S. application Ser. No. 10/822,081 filed on Apr. 12, 2004; U.S. application Ser. No. 11/242,642 filed on Oct. 3, 2005 and U.S. application Ser. No. 11/302,654 filed on Dec. 14, 2005, all commonly owned by the present assignee.

The control between the proximal bendable member 18 and distal bendable member 20 is provided by means of the bend control cables 100. In the illustrated embodiments four such control cables 100 are provided in order to provide the desired all direction bending. However, in other embodiments of the present invention fewer or more numbers of bend control cables may be used. The bend control cables 100 extend through the instrument shaft 14 and through the proximal and distal bendable members. The bend control cables 100 are preferably constrained along substantially their entire length so as to facilitate both “pushing” and “pulling” action. The cables 100 are also preferably constrained as they pass over the conical cable guide portion 19 of the proximal bendable member, and through the proximal bendable member.

The locking means of the present invention, rather than using a ball and socket arrangement to lock and unlock the instrument, uses a cable or wire scheme in association with a locking ring. This lock control allows the surgeon two less degree of freedom (orthogonal bending) to concentrate on when performing certain tasks. By locking the bendable sections at a particular position, this enables the surgeon to be more hands-free for controlling other degrees of freedom of the instrument such as manipulation of the rotation knob 24 and, in turn, orientation of the end effector.

The instrument shown in FIG. 1 is of a pistol grip type. However, the principles of the present invention may also apply to other forms of handles such as a straight in-line handle. In FIG. 1 there is shown a jaw clamping means 30 that is comprised mainly of the lever 22 which has a single finger hole 23 defined by the supported gimbal ball 27, for controlling the lever. The lever 22 preferably also includes a related release function that may either be controlled directly by the lever 22 or by a separate release button. The release function is used to release the actuated or closed tool.

In the instrument that is illustrated the handle end of the instrument may be tipped in any direction as the proximal bendable member is constructed and arranged to enable full 360 degree bending. This movement of the handle relative to the instrument shaft bends the instrument at the proximal bendable member 18. This action, in turn, via the bend control cables 100, bends the distal bendable member in the same direction. As mentioned before, opposite direction bending can be used by rotating or twisting the control cables through 180 degrees from one end to the other end thereof.

In the embodiment described herein, the handle 12 is in the form of a pistol grip and includes a horn 13 to facilitate a comfortable interface between the action of the surgeon's hand and the instrument. The tool actuation lever 22 is shown in FIG. 1 pivotally attached at the base of the handle. The lever 22 actuates a slider (not shown) that controls a tool actuation cable 38 that extends from the slider to the distal end of the instrument. The cable 38 controls the opening and closing of the jaws 44, 46, and different positions of the lever control the force applied at the jaws. Refer to co-pending U.S. application Ser. No. 11/528,134 filed on Sep. 27, 2006 and hereby incorporated by reference herein in its entirety, for further details of the jaw clamping means 30 and associated handle mechanism.

The shape of the handle allows for a comfortable and substantially one-handed operation of the instrument as shown in FIG. 1. As shown in FIG. 1, the surgeon may grip the handle 12 between his palm and middle finger with the horn 13 nestled in the crook between his thumb and forefinger. This frees up and positions the forefinger and thumb to rotate the rotation knob 24 using the finger indentions 31 that are disposed on the peripheral surface of the rotation knob, as depicted in FIG. 1. In both locked and unlocked positions of the instrument the rotation knob is capable of controlled rotation to control axial rotation at the tip of the instrument about the distal tool tip axis P, as represented by the rotation arrow R3 in FIG. 1. This rotation can occur regardless of the orientation (angle of the axis P).

In the disclosed embodiment there is provided at the tool closing lever 22 a fingertip engaging recess 23 in a gimbaled ball 27. The free end of the lever 22 supports the gimbaled ball 27 which has a through hole or recess 23 which receives one of the fingers of the user. The ball 27 is free to at least partially rotate in three dimensions in the lever end. The surgeon may grip the handle between the palm, ring and pinky fingers with the horn 13 nestled in the crook between his thumb and forefinger and operate the rotation knob 24 as previously described. The surgeon may then operate the jaw clamping lever 22 with the forefinger or middle finger.

The gimbal is in the form of a ball in a socket, in which the ball 27 is free to be rotated in the socket, and in which the socket is defined in the lever free end. In this embodiment, rather than having the hole or recess 23 go completely through the ball there may be provided a blind hole in the ball. The ball is free to rotate in the lever end and thus the ball can also be rotated to alternate positions corresponding to either a right-handed or left-handed user. The blind hole (in comparison to a through hole) enables the user to have a firmer grip of the lever and thus enhanced control of the lever action.

In this instrument the distal bendable member 20 is shown in FIG. 1 with a protective sheath 98. The distal bendable member may be comprised of spaced discs that define therebetween spaced slots. Ribs may connect between adjacent discs in a manner similar to that described in the afore-mentioned U.S. application Ser. No. 11/185,911. The distal bendable member may be substantially the same as the illustrated proximal bendable member, but preferably of smaller diameter.

The end effector 16 is comprised of a pair of jaws 44 and 46. The jaws 44 and 46 may be used to grasp a needle or other item. The upper jaw 44 preferably fits within a channel in the lower jaw 46. A pivot pin may be provided between the jaws to enable rotation therebetween. When the lever 22 is in its rest position, the jaws are fully open. In that position the control pin is at a more distal location maintaining the jaws in an open position. As the cable 38 is pulled, then the pin moves to the right causing the jaws 44 and 46 to pivot toward a closed position.

The rotation knob 24 is provided with a proximal hub 25 which supports the proximal end of the proximal bending member 18. During assembly, the cables 100 which protrude from the proximal end of the proximal bending member 18, after the assembly of the end effector 16, inner and outer shafts 32, 34, adapter 26 and proximal bending member 18, are passed through the four terminal wire crimps or lugs 102 which are keyed into passages in the hub 25. The cables are tensioned and crimped and excess cable material is trimmed off. This arrangement holds all the elements together between the end effector 16 and the rotation knob hub 25 and, in turn, the rotation knob 24.

As indicated previously, the rotation knob 24 is formed with a hub 25 on its proximal side that is supported on the center wire conduit 64 which extends from the rotation knob 24 back to the slider. An e-ring 65 may retains the hub 25 in a rotational relationship relative to the conduit 64. The conduit 64 is supported in a fixed position by internal means of the handle 12. The knob 24 is readily accessible through a gap 232 between the hub 202 and the distal end of the handle. See the gap 232 in FIG. 2. The rotation knob 24 may be provided with four keyhole shaped slots that receive terminal wire crimps 102.

As indicated previously, the end effector or tool 16 is actuated by means of a jaw actuation mechanism or jaw clamping means 30 which is comprised primarily of the elongated lever 22. The lever 22 is supported from the housing at the lever pivot pin. The closing of the lever 22 against the handle 12 acts upon the slider (not shown) which is used to capture the very proximal end of the actuation cable 38. In one position the end effector jaws are fully open. In that position the slider is disposed at the more distal end of its slideway. The slideway (not shown) is part of the internal support in the handle 12. When the slider is moved proximally, then the jaws are moved toward a closed position.

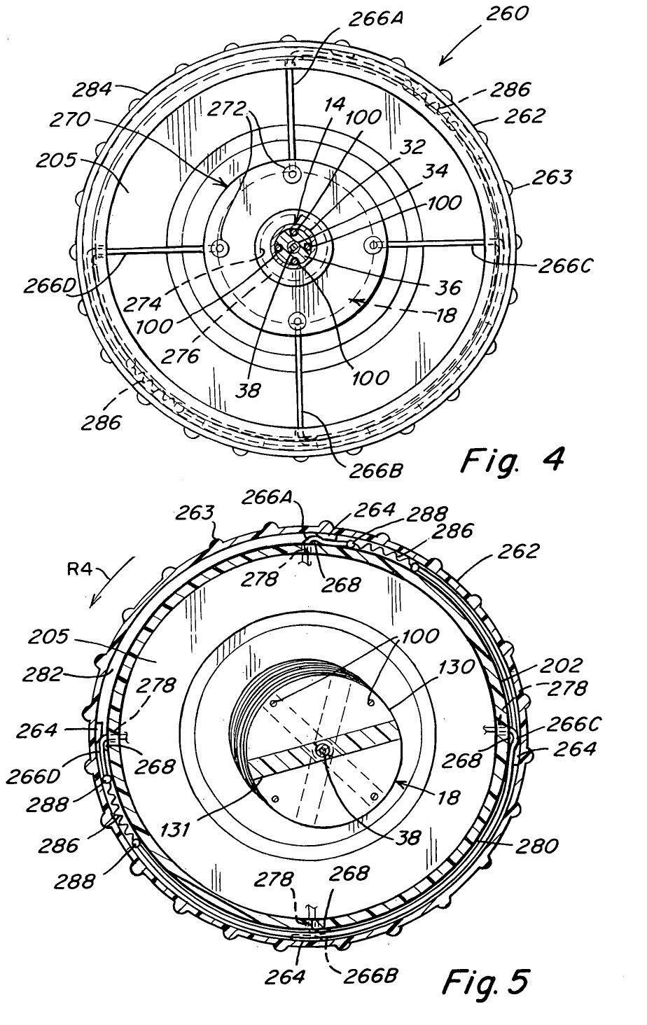

The instrument shaft 14 includes an outer shaft tube 32 that may be constructed of a light weight metal material or may be a plastic material. See the cross-sectional view of FIG. 4 taken through the instrument shaft. The proximal end of the tube 32 is received by the adaptor 26, as depicted in FIGS. 2 and 3. The distal end of the tube 32 is secured to the distal bendable member 20. Within the outer shaft tube 32 there is provided a support tube 34 that is preferably constructed of a plastic material. Tube 34 extends between the distal bendable or flexible member 20 and the proximal bendable or flexible member 18. The jaw actuator cable 38 extends within this support tube 34.

One of the features of the present invention is the cable scheme that uses bend control cables that are relatively stiff and yet are bendable. The stiffer cables allow for, not only “pulling”, but also “pushing” action thereof. This enables enhanced control via the cabling as control is provided, not only when a cable is “pulled”, but also when a cable is “pushed”. This makes for a more uniform control via the cables. To enable, not only a “pulling” action, but also a “pushing” action, the cables 100 are supported in relatively narrow lumens or passageways to prevent buckling when being pushed. This is facilitated by, inter alia, the provision of a shaft filler 36. To allow for the “pushing” action in particular the cables are confined so that they do not distort within the instrument itself.

The shaft filler 36 is disposed between the tubes 32 and 34 and is used to hold the cables in place within the instrument shaft itself. The shaft filler has a central lumen for the inner shaft support tube 34 and may be provided with four lengthwise grooves that accommodate and allow a snug sliding fit for the cables 100. The conical portion 19 of the proximal bending member 18 also may have four cable guide grooves disposed at 90 degree intervals about its outer surface that capture each cable in a sliding relationship with the adapter 26. Each of the guide grooves is may be formed in a separate diametrically disposed wing of the conical portion 19. The adaptor 26 may also be provided with accommodating grooves for the cables 100. Thus, the cables are constrained along their length in grooves or passages. Each of the cables is preferably unsupported for only a short distance such as the distance of the slots 132, or like slots at the distal bendable member.

The jaw actuator cable 38 terminates at its respective ends at the end effector and a rotation barrel (not shown). Within each of the bendable sections or bendable members 18 and 20 there is provided a plastic tube. This includes a distal tube and a proximal tube. Both of these tubes may be constructed of a plastic such as polyethyletherkeytone (PEEK). The material of the tubes is sufficiently rigid to retain the cable 38 and yet is flexible enough so that it can readily bend with the bending of the bendable members 18 and 20. The tubes have a sufficient strength to receive and guide the cable, yet are flexible enough so that they will not kink or distort, and thus keep the cable in a proper state for activation, and also defines a fixed length for the cable. The tubes are longitudinally stiff, but laterally flexible.

The proximal bendable member 18, like the distal bendable member 20, may also be constructed as a unitary or uni-body slotted structure including a series of flexible discs 130 that define therebetween slots 132, as shown in FIG. 2. A “unitary” or “uni-body” structure may be defined as one that is constructed for use in a single piece and does not require assembly of parts. Connecting ribs 131 are illustrated as extending between adjacent discs 130. Both of the bendable members preferably have a rib pattern in which the ribs are disposed at a preferred 60 degree variance from one rib to an adjacent rib. This has been found to provide an improved bending action. It was found that by having the ribs disposed at intervals of less than 90 degrees therebetween improved bending was possible. The ribs may be disposed at intervals of from about 35 degrees to about 75 degrees from one rib to an adjacent one. By using an interval of less than 90 degrees the ribs are more evenly distributed. As a result the bending motion is more uniform at any orientation. In the present invention both of the bendable members may be made of a highly elastic polymer such as PEBAX (Polyether Block Amide), but could also be made from other elastic and resilient materials.

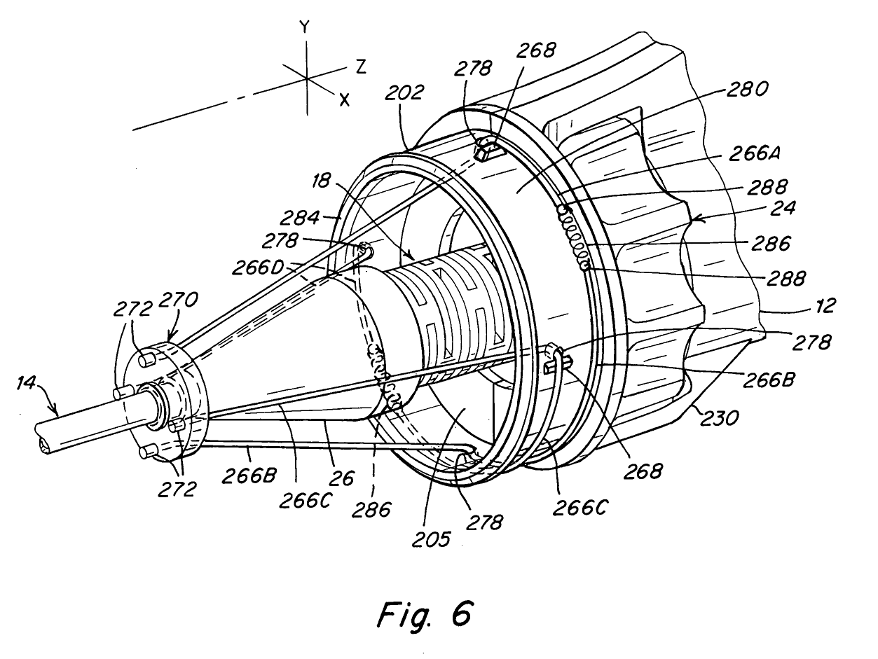

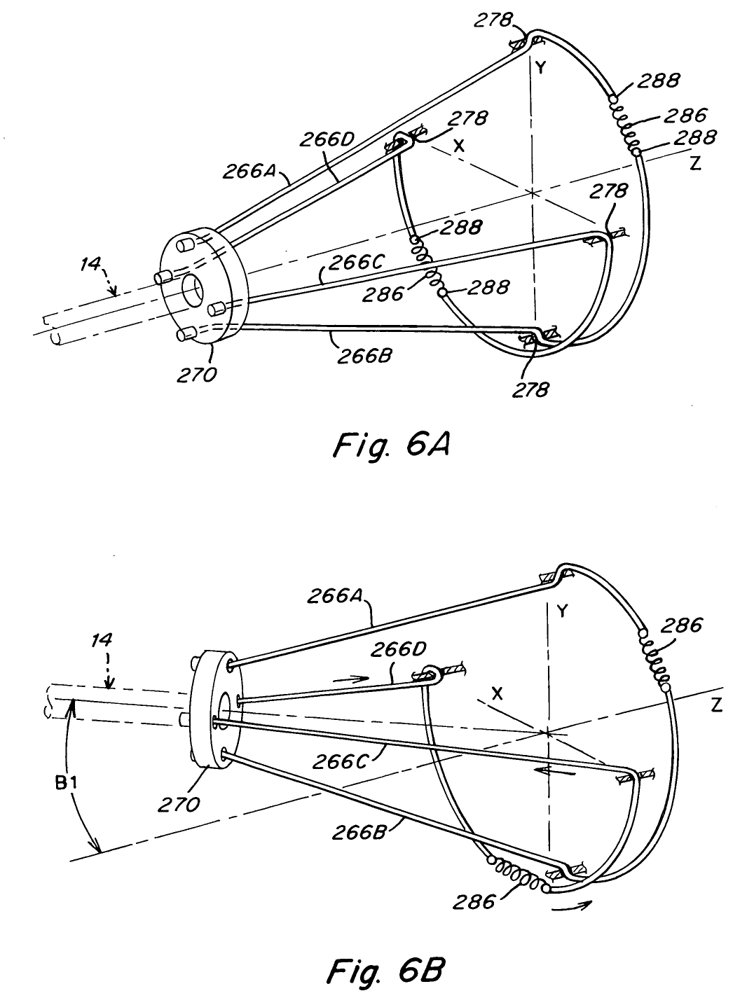

Reference is now made to a first embodiment of the present invention that employs a cable scheme for locking the instrument. This enables a somewhat smaller locking mechanism, and yet one that is quite effective in enabling the surgeon to lock the position of the instrument at a desired position. This first embodiment is shown in FIGS. 1-6 including the schematic diagrams in FIGS. 6A and 6B. The locking mechanism or angle locking means 260 includes a cable arrangement that is basically disposed over the proximal bendable member and that follows the bending at the proximal bendable member. The locking mechanism has locked and unlocked positions, is disposed about the proximal movable or bendable member and is manually controlled so as to fix the position of the proximal movable member relative to the handle in the locked position thereof. The locking mechanism also includes a locking ring that is rotatable between locked and unlocked positions.

With regard to the first embodiment, FIG. 1 is a perspective view of this first embodiment of the present invention. FIG. 2 is a fragmentary cross-sectional side view of the instrument of FIG. 1. FIG. 3 is a cross-sectional view similar to FIG. 2 but showing the instrument shaft in an angled position. FIG. 4 is a cross-sectional view taken along line 4-4 of FIG. 2. FIG. 5 is a cross-sectional view taken along line 5-5 of FIG. 3. FIG. 6 is a fragmentary perspective view of the embodiment of FIG. 1 with the locking ring removed for simplicity. FIG. 6A is a schematic perspective view of the cabling mechanism of FIG. 6. FIG. 6B is a schematic perspective view similar to that shown in FIG. 6A but with the instrument rotated 35 degrees in the “X” direction.

In this first embodiment the proximal bendable member 18 is shown supported between the rotation knob 24 and the instrument shaft 14. This support includes the adapter 26 as an interface between the instrument shaft 14 and the conical portion 19 of the proximal bendable member 18. The distal end of the adapter 26 supports the anchor disc 270, as shown in FIGS. 2 and 3. It is the anchor disc 270 that provides the fixed support for the cabling 266. Refer to FIGS. 6A and 6B which show the anchor ring or disc 270 with all four cable ends fixedly attached at the anchor disc 270.

At the handle end, the cabling 266 is supported essentially between the outer surface 280 of the hub 202, and the locking ring 262. Refer to FIG. 6 where the locking ring has been removed to show the surface 280 and certain sections of the cabling extending about the surface 280. It is the interaction between the locking ring, and in particular the rotation thereof, and the hub outer surface that provides the locking/unlocking action. This is caused by a pinching the cabling 262 to lock position. In FIG. 1 a flexible sheath is illustrated covering the proximal bendable member and cabling.

The locking means 260 is engaged by rotating the locking ring 262 in direction of arrow R4 as depicted in FIG. 5. The locking ring 262 has an outer gripping surface including ribs 263 and is retained on the hub 202 by means of the bezel 284. The locking ring 262 is dimensioned so as to provide a passageway 282 between the locking ring and the hub 202. The passageway 282 accommodates the sliding cables 266 and the associated springs 286. FIG. 5 shows the passageway 282 and the springs 286 disposed therein. FIG. 6 shows the passageway opened up and the manner in which the cables 266 and springs 286 are run over the hub surface 280. The locking mechanism ring 262 is reachable to the user by using the thumb and forefinger to enable rotation thereof by the user of the instrument.

The inner surface of the locking ring 262 is also provided with four semi-resilient internal cams 264 that engage and pinch tight the cables 266A-D. Refer to the cross-sectional view of FIG. 5 that depicts the cams 264 that are disposed at 90 degree interval about the inner surface of the locking ring 262. To interact with these cams, the hub 202 is provided, on its outer surface, with ribs 268 that are fixed in position on the hub and also disposed at 90 degree intervals about the hub 202. FIG. 5 shows the close proximity of the cam and rib, and in FIG. 5 the angle locking is in a locked position wherein the cable 266 is pinched between the cam 264 and the rib 268. This is caused by the rotation of the locking ring 262, in the direction of arrow R4 in FIG. 5. This action effectively locks in the angle of the instrument shaft.

Reference may now be made to FIG. 6, as well as the schematic diagrams shown in FIGS. 6A and 6B, for an illustration of the manner in which the cabling 266 is supported and operated. The cabling has been identified as cable sections 266A, 266B, 266C and 266D. The cabling is actually formed by two separate loops. One loop includes sections 266A and 266B, and the other loop includes sections 266C and 266D. A spring 286 is disposed between these sections, as shown in the schematic diagrams. The set of cables 266 is fixedly attached to the anchor disk 270 by crimps 272. The cabling also passes through four passageways 278 in the hub 202 and wrap around the outer surface 280 of the hub 202. The cables 266A-266D are connected at their proximal ends to springs 286 by means of the terminals 288. The cables are connected to each other in the X & Y axes so that bending of the shaft in one direction will result in one cable being played out from the hub and the opposing cable being reeled in with the spring 286 as a buffer between cable sections.

In the perspective view of FIG. 6 the positioning of the cabling is illustrated. Cable section 266A is connected in the Y axis direction to cable section 266B by a first spring 286 and similarly cable section 266C is connected to cable section 266D by a second spring 286, but in the X axis direction. The springs 286 can be positioned approximately midway between passageways 278 where they have clearance to slide past each other and enough unimpeded movement to allow the cables 266A-266D to fully play out from the hub when the shaft reaches a maximum bend angle B1. The springs 286 keep tension on the cables and allow for a small discrepancy between the lengths of cable sections playing in and out as the shaft of the instrument is bent at various angles. As the shaft is angled the cabling simply transitions along the hub surface 280 passing through the passageways 278. The anchor disk 270 has a bearing surface 274 that seats in raceway 276 on the adapter 26 as seen in FIG. 2, and that allows the shaft and adapter to rotate freely when the rotation knob 24 is rotated. The proximal bendable member 18 and cabling 266 are preferably sheathed in a flexible bellows member 290, as best illustrated in FIG. 1. The bellows member 290 may extend around the anchor disk 270 and may be attached at bezel the 284. For the sake of simplicity the sheath is shown in phantom line in the figures other than FIG. 1.

Reference is now made to the schematic diagrams of FIGS. 6A and 6B. FIG. 6A shows the position of the cabling when the instrument shaft is in a straight position relative to the handle of the instrument. In other words the angle B1 is zero degrees. In that position the springs 268 may be disposed about midway of the passageways 278. FIG. 6B is a schematic perspective view similar to that shown in FIG. 6A but with the instrument rotated 35 degrees in the “X” direction. In actual practice the deflection angle is usually a composite of both “X” and “Y” coordinates movement. This is a rotation indicated by the angle B1 in FIG. 6B. Refer in FIG. 6B also to the directional arrows that illustrate how the cable sections are moved through the passageways 278 to re-position the cable sections. When the locking ring is then rotated, that pinches the cable sections at that location locking the position of the instrument.

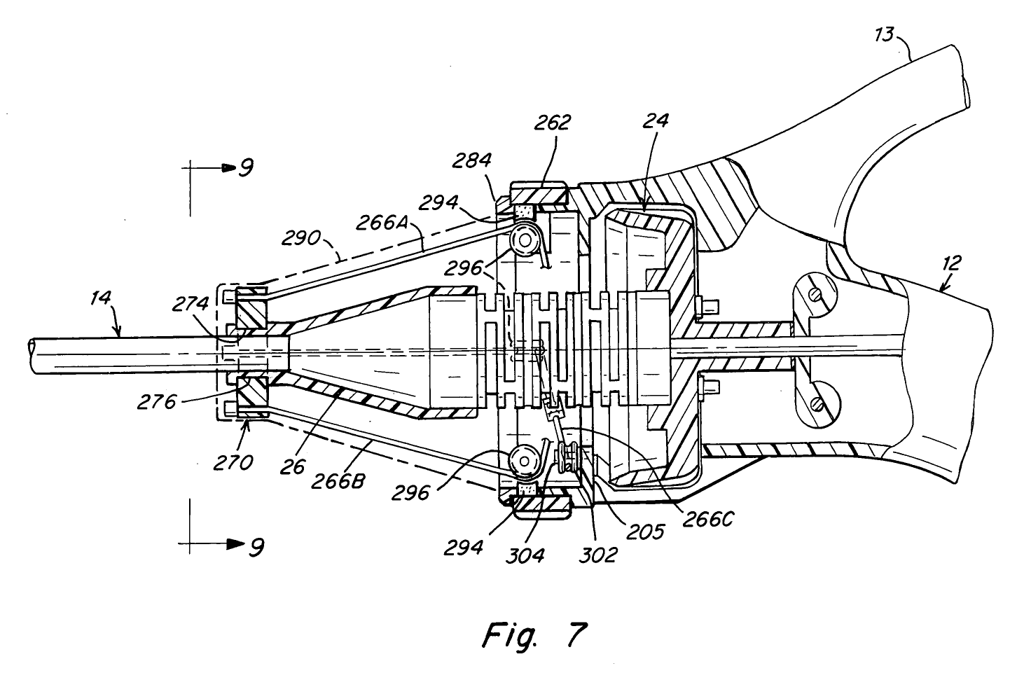

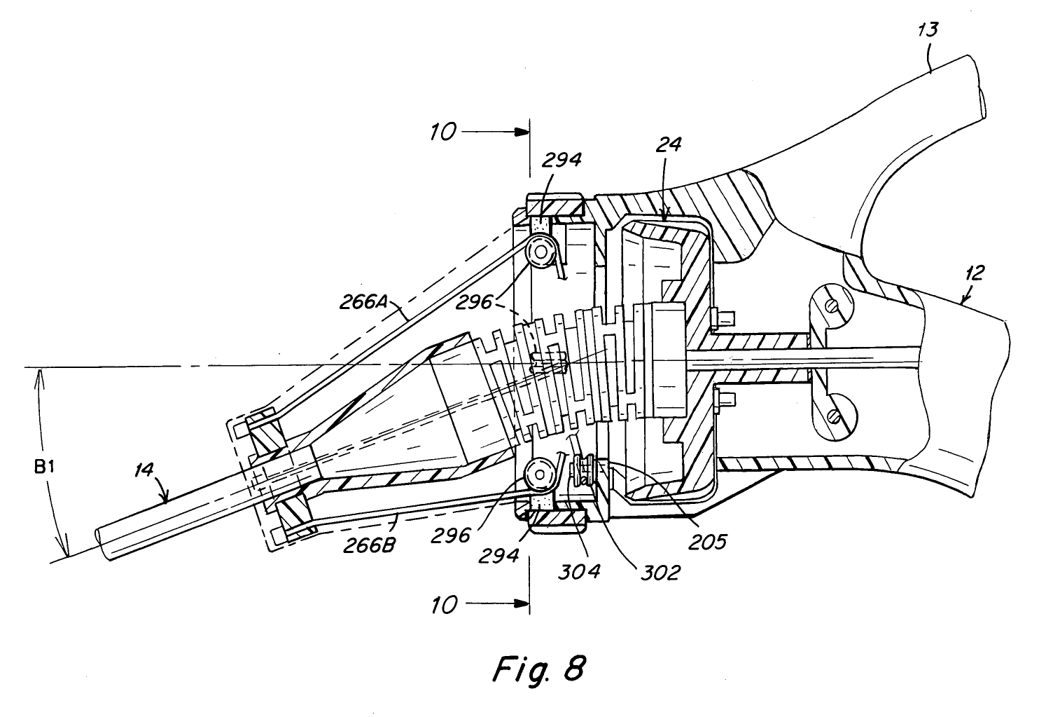

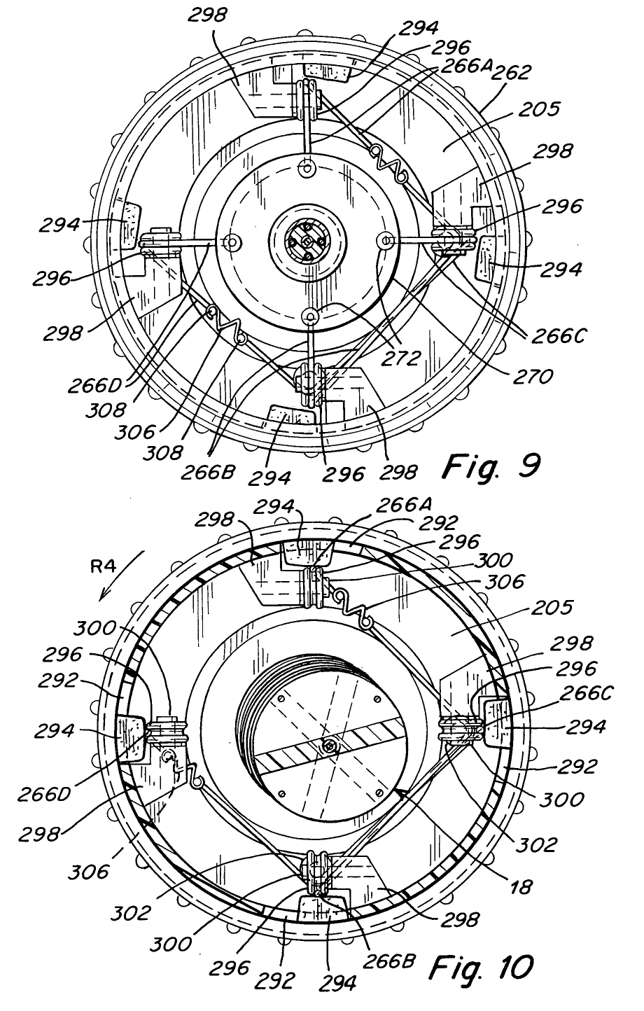

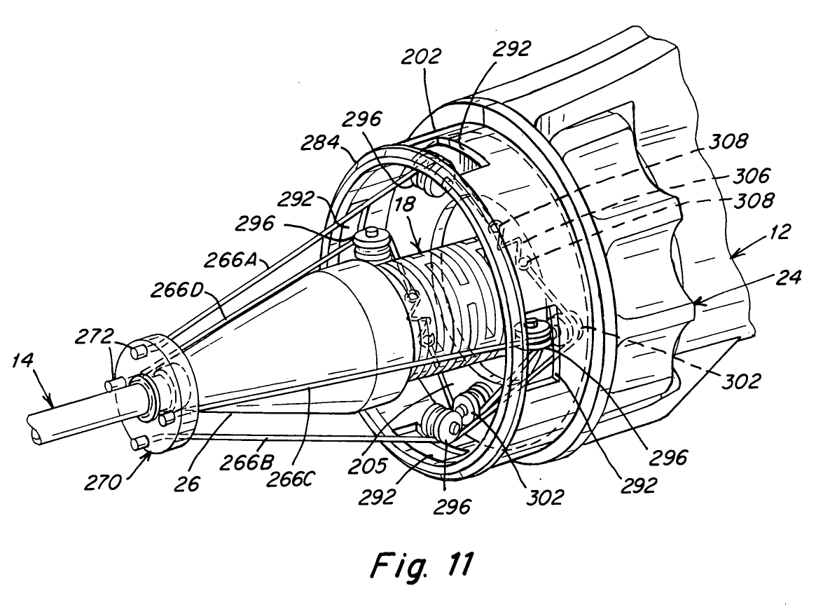

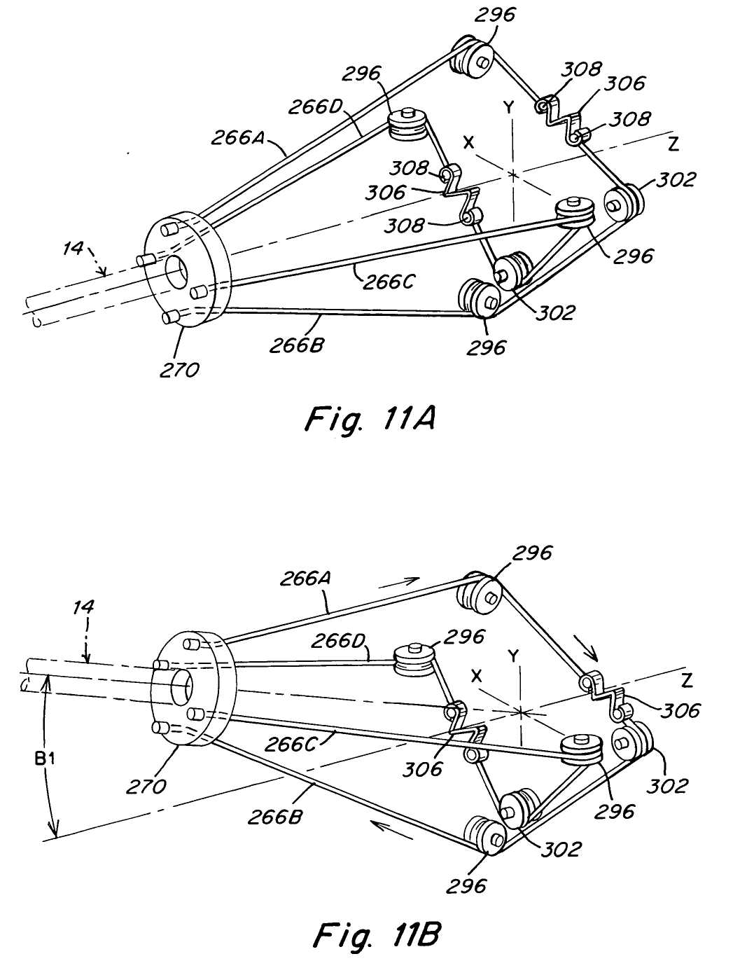

A second embodiment of the present instrument is shown in FIGS. 7-11. FIG. 7 is a fragmentary cross-sectional side view of this second embodiment. FIG. 8 is a cross-sectional view similar to that shown in FIG. 7 but illustrating the instrument shaft in an angled position. FIG. 9 is a cross-sectional view taken along line 9-9 of FIG. 7, while FIG. 10 is a cross-sectional view taken along line 10-10 of FIG. 8. FIG. 11 is a fragmentary perspective view of the instrument of FIG. 7 with the locking ring removed for simplicity of description. FIG. 11A is a schematic perspective view of the cabling mechanism of FIG. 11. FIG. 11B is a schematic perspective view similar to that shown in FIG. 11A but with the instrument rotated 35 degrees in the “Y” direction.

For the second embodiment described in FIGS. 7-11 many of the same reference numbers are used, as in the first embodiment to describe like components. In this second embodiment, a series of pulleys 296 are used to reduce the friction of the cable rubbing against passageways 278, as well as to reduce the sliding action of the cabling and springs against the outside surface 280 of the hub 202. This also allows the cabling and springs to be essentially suspended inside the hub 202, making for a compact arrangement. The locking occurs by interaction between the locking ring 262 and the pulleys 296, as is described in more detail hereinafter.

In this second embodiment, the cables 266 extend from the anchor disk 270 to and over pulleys 296 which are mounted to posts 298 by means of the axles 300. In this regard refer to the cross-sectional views of FIGS. 9 and 10 that illustrate the 90 degree placement of the posts 298. The cable pairs 266A and 266B and the pairs 266C and 266D then pass around a second set of pulleys 302 which are mounted on axles 304 attached to a radial wall 205 of the hub 202. In this regard refer to FIG. 7 which shows the position of the pulley 302 relative to that of the pulley 296. Refer also to FIG. 10 that illustrates both pulleys, with pulleys 302 disposed behind pulleys 296. The cable pairs are connected by springs 306 at end terminals 308 (FIG. 11A). In this embodiment the locking ring 262 has four semi-resilient internal cams 294 that pass through clearance slots 292, as depicted, for example, in FIGS. 10 and 11. These cams 294 pinch the cables 266A-266D against the pulleys 296 and also engage the rims of the pulleys 296 when the locking ring 262 is rotated in the direction of arrow R4. FIG. 9 shows the unlocked or released position where the cams are separated from the pulleys while FIG. 10 shows the locked position wherein the cams 294 are in contact with the cable against the associated pulley.

Reference may now be made to FIG. 11, as well as the schematic diagrams shown in FIGS. 11A and 11B, for an illustration of the manner in which the cabling 266 is supported and operated. The cabling has been identified as cable sections 166A, 166B, 166C and 166D. The cabling is actually formed by two separate loops. One loop includes sections 266A and 266B, and the other loop includes sections 266C and 266D. A spring 306 is disposed between these sections, as shown in the schematic diagrams. The set of cables 266 is fixedly attached to the anchor disk 270 by crimps 272. The cabling also passes over the aforementioned pulleys. The cables 266A-266D are connected at their proximal ends to springs 306 by means of the end terminals 308. The cables are connected to each other in the X & Y axes so that bending of the shaft in one direction will result in one cable being played out from the hub and the opposing cable being reeled in with the spring 306 as a buffer between cable sections.

In the perspective view of FIG. 11 the positioning of the cabling is illustrated. Cable section 266A is connected in the Y axis direction to cable section 266B by a first spring 306 and similarly cable section 266C is connected to cable section 266D by a second spring 306, but in the X axis direction. The springs 306 can be positioned approximately midway between pulley 296 and pulley 302, as shown in the straight position of the shaft in FIG. 11A. The springs 306 keep tension on the cables and allow for a small discrepancy between the lengths of cable sections playing in and out as the shaft of the instrument is bent at various angles. As the shaft is angled the cabling simply transitions about the support pulleys. The anchor disk 270 has a bearing surface 274 that seats in raceway 276 on the adapter 26 as seen in FIG. 7, and that allows the shaft and adapter to rotate freely when the rotation knob 24 is rotated. The proximal bendable member 18 and cabling 266 are preferably sheathed in a flexible bellows member 290, as illustrated previously in the first embodiment in FIG. 1. The bellows member 290 may extend around the anchor disk 270 and may be attached at bezel the 284. For the sake of simplicity the sheath is shown in phantom line in the figures other than FIG. 1.

Reference is now made to the schematic diagrams of FIGS. 11A and 11B. FIG. 11A shows the position of the cabling when the instrument shaft is in a straight position relative to the handle of the instrument. In other words the angle B1 is zero degrees. In that position the springs 306 may be disposed about midway between the pulleys 296 and 302. FIG. 11B is a schematic perspective view similar to that shown in FIG. 11A but with the instrument rotated 35 degrees in the “Y” direction. In actual practice the deflection angle is usually a composite of both “X” and “Y” coordinates movement. This is a rotation indicated by the angle B1 in FIG. 11B. Refer in FIG. 11B also to the directional arrows that illustrate how the cable sections are moved over the pulleys to re-position the cable sections. When the locking ring is then rotated, that pinches the cable sections at that location locking the position of the instrument.

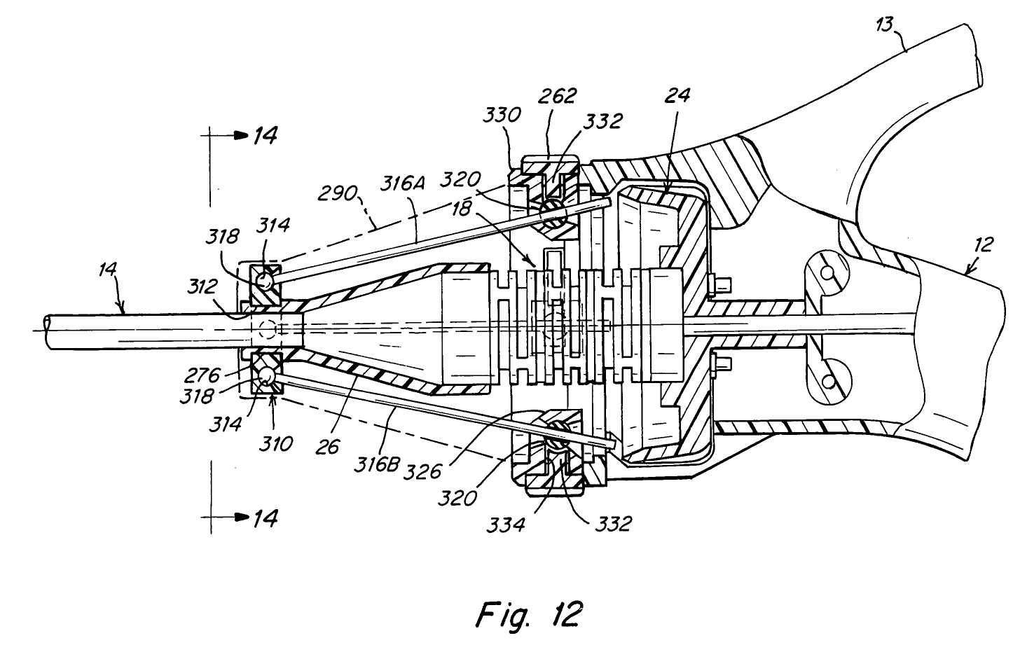

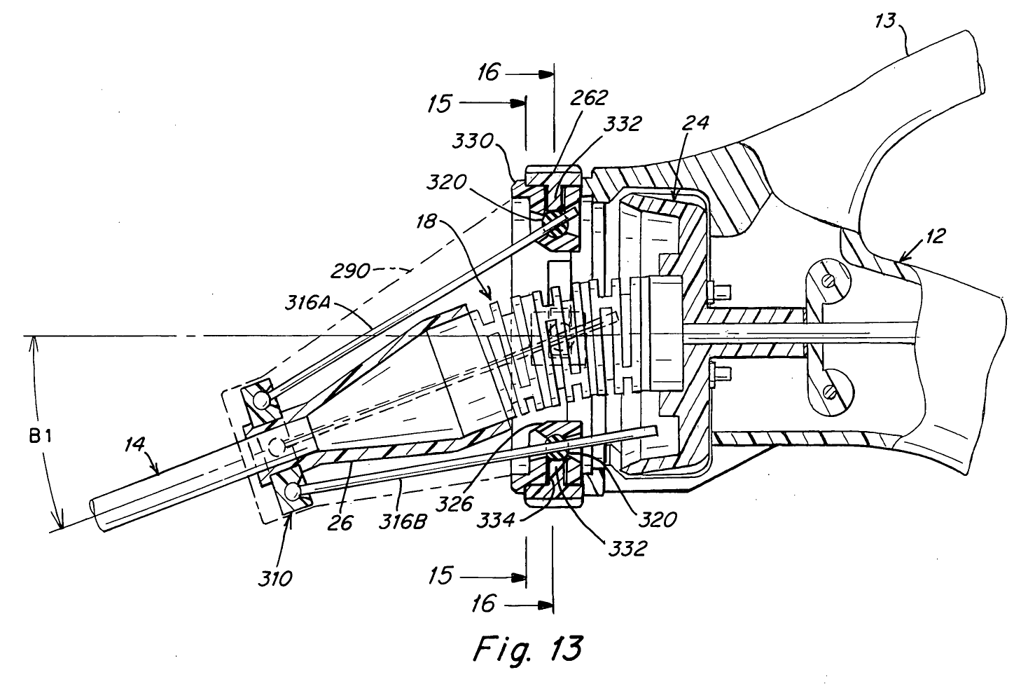

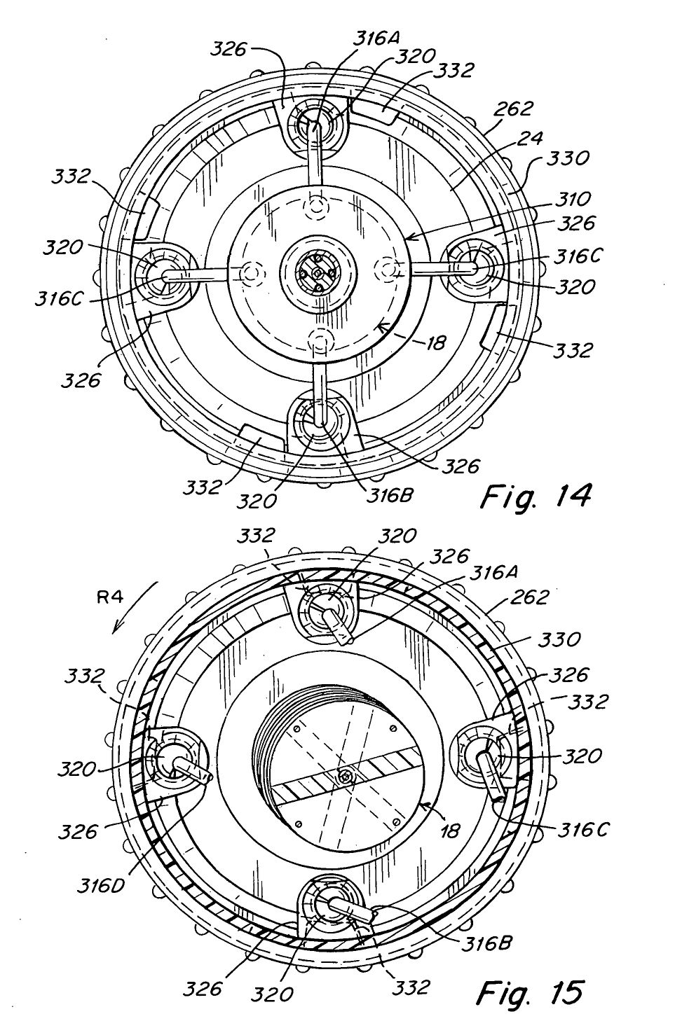

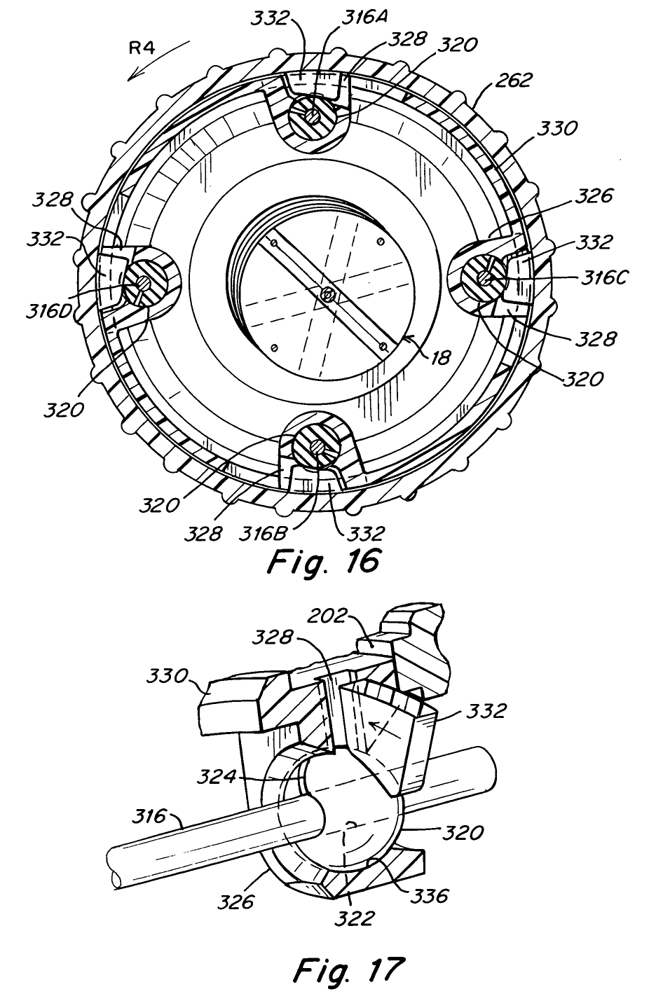

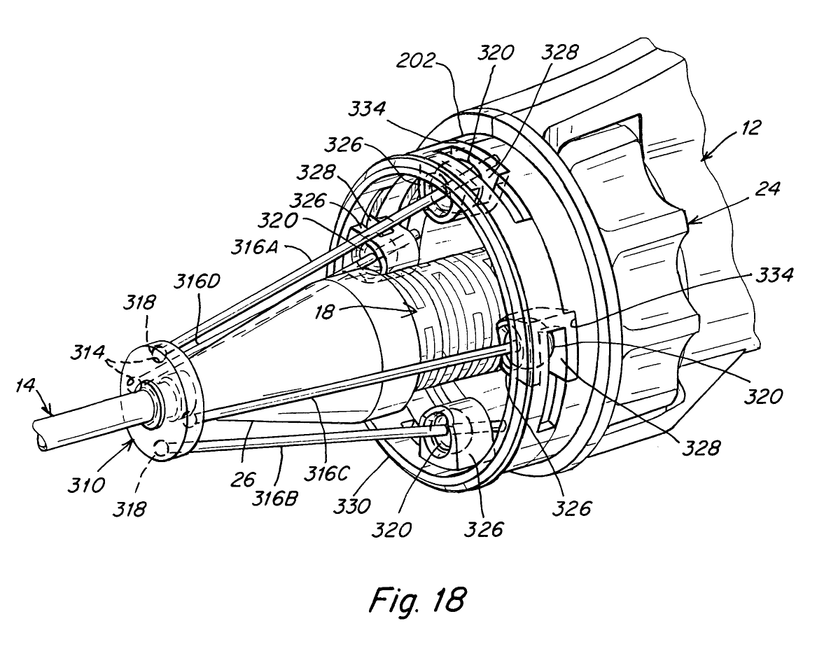

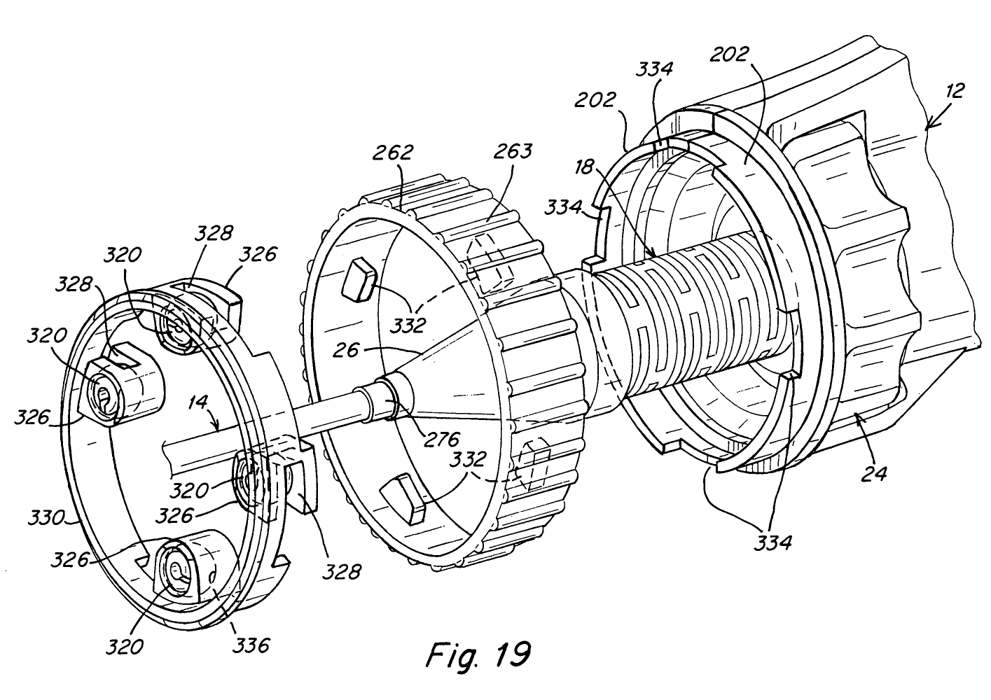

A third embodiment of the present instrument is shown in FIGS. 12-19. FIG. 12 is a fragmentary cross-sectional side view of this third embodiment. FIG. 13 is a cross-sectional side view similar to that shown FIG. 12 but with the instrument shaft in an angled position. FIG. 14 is cross-sectional view taken along line 14-14 of FIG. 12. FIG. 15 is a cross-sectional view taken along line 15-15 of FIG. 13. FIG. 16 is a cross-sectional view taken along line 16-16 of FIG. 13. FIG. 17 is a fragmentary enlarged detail perspective view of a locking cam arrangement for the embodiment of FIG. 12. FIG. 18 is a fragmentary perspective view of the embodiment of FIG. 12 with the locking ring removed for simplicity of description. FIG. 19 is an exploded perspective view of the embodiment shown in FIG. 12. For the third embodiment described in FIGS. 12-19 many of the same reference numbers are used, as in the previous embodiments to describe like components.

In the first two embodiments that are described herein the locking feature includes the use of cabling that is either passed over the hub outer surface or about pulleys. In the third embodiment of the instrument, as shown in FIGS. 12-19, the cables 266 have been replaced with rigid rods 316 which provide a stiffer angle locking arrangement since, not only are the lengths of the rods 316 locked in place, but also the angles of the rods 316 are locked in place. This, along with the use of an anchor disc 310, provides a stable platform for the locking feature. The locking is provided primarily by the locking ring 262 which is of somewhat different construction than the locking rings described in the earlier embodiments. The locking ring 262 also uses a cam arrangement for locking the rigid rods at a locked position.

Each of the rigid rods 316A-316D has a proximal free end and an end pivot ball 318 at the opposite end. The anchor disk 310 has four spherical sockets 314 that receive the respective balls 318. Refer to the perspective view of FIG. 18 for an illustration of the rigid rods 316 and the maimer in which the ball ends thereof are held in their respective sockets 314. The balls 318 are supported with limited rotation in each socket to accommodate the bending action, such as is shown in FIG. 13. The anchor disc 310 also is provided with a bearing surface 312 that rides in the raceway 276 in the adapter 26. The proximal ends of the rods 316 pass through passages 322 in split balls 320 which are, in turn, mounted in housings 326. Each of the housings 326 is disposed at 90 degree intervals about the bezel ring 330 as shown in FIG. 19. The housings 326 are mounted to the bezel ring 330 which is, in turn, mated to the hub 202. Refer also to the fragmentary perspective view of FIG. 17 that illustrates the split ball 320 with its split at 324, along with the bezel ring 330, hub 202 and cam 332.

As illustrated in FIGS. 18 and 19, the housings 326 each mate with a corresponding slot 334 in hub 202. These slots are also disposed about the hub at 90 degree intervals. Each of the housings is provided with a slot 328 that receives the cams 332. The cams 332 are also disposed at and affixed at an inner surface of the locking ring 262 as depicted in FIG. 19. The slots 328 in the housings 326 allow the cams 332, disposed on the inside surface of locking ring 262, to engage and compress the split balls 320 when the locking ring is rotated in the direction of arrow R4, as shown in FIGS. 15 and 16. The bezel ring 330 may be welded or otherwise fastened to the hub 202 and constrains the locking ring 262 on the hub 202. As can be seen in FIG. 17 the proximal ends of rods 316 are free to slide within passageways 322 in each respective split ball 320 when the locking ring is in its unlocked position. This position is illustrated in the cross-sectional view of FIG. 14 wherein it is to be noted that the locking cams 332 are spaced apart from the split balls 320. It is moreover noted that the split balls 320 are free to pivot or rotate in their respective spherical sockets 336 in housings 326. When the locking ring is rotated in the direction of arrow R4 the cams 332 compress the split balls, clamping the balls against rods 316 and against the housings 326. In this regard refer to the cross-sectional view of FIG. 16 which illustrates the cam 332 compressing the split ball 320. This locks in the lengths and the angles of the rods 316 relative to the hub 202 and thus the position of anchor disk 310 and angle B1.

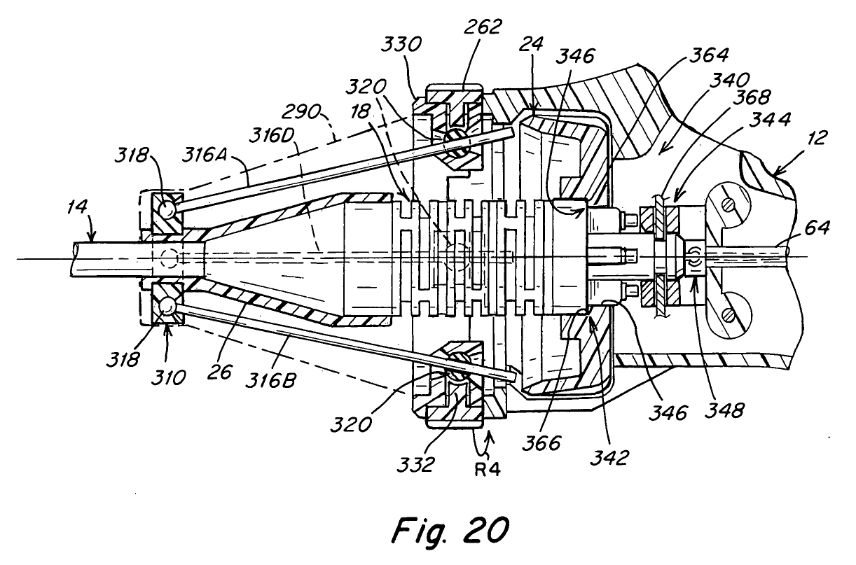

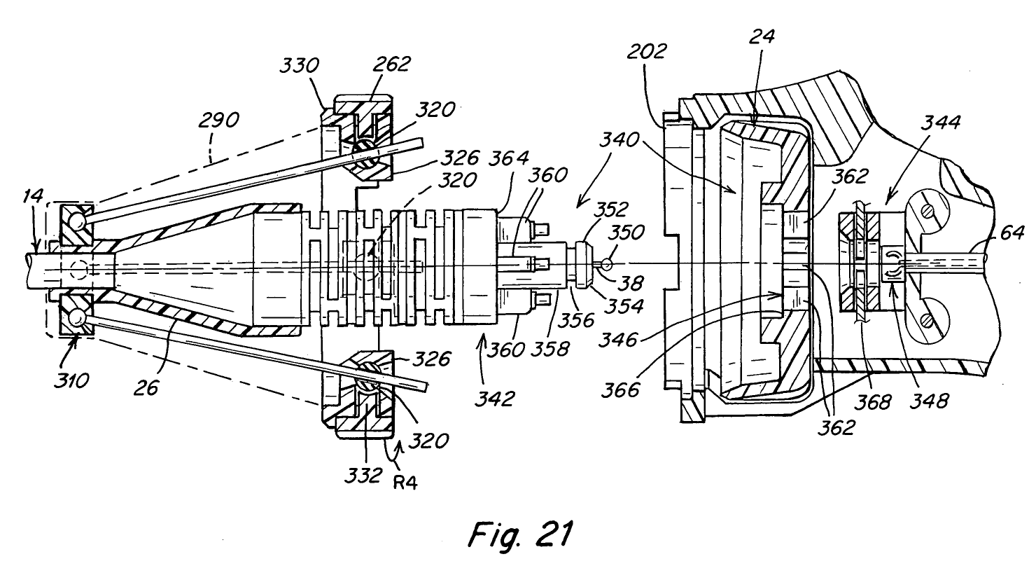

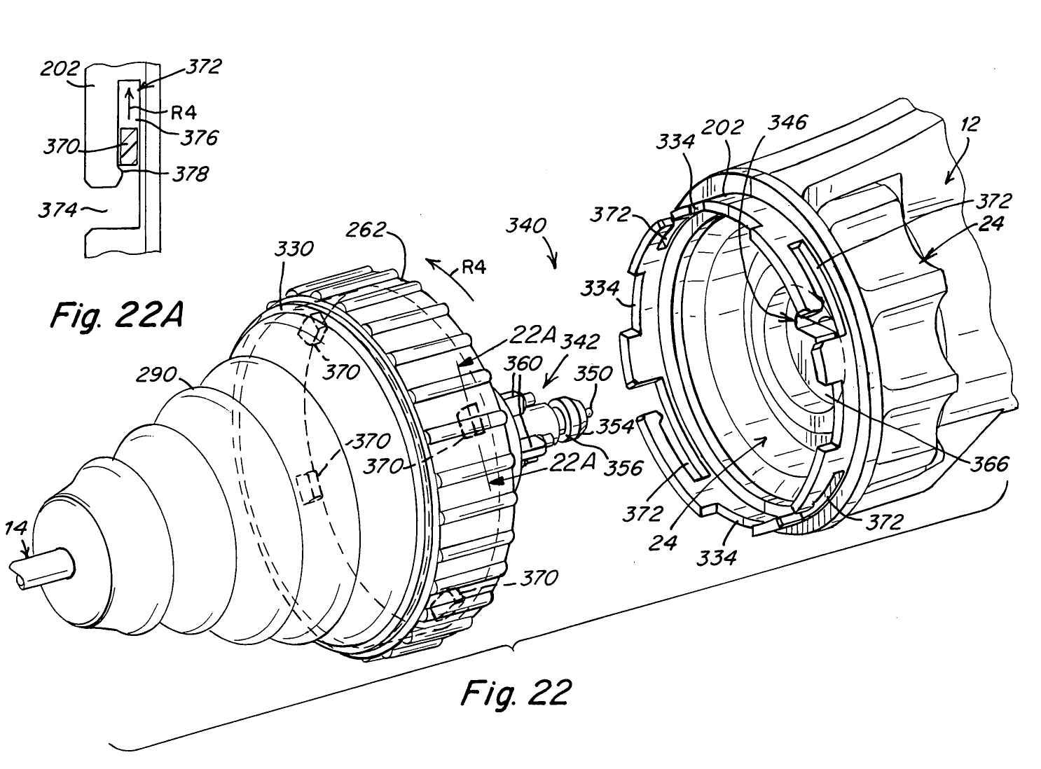

A fourth embodiment of the instrument is shown in FIGS. 20-22 wherein at least parts of the instrument are considered as disposable or reposable. Regarding this embodiment FIG. 20 is a fragmentary cross-sectional side view of this fourth embodiment. FIG. 21 is a cross-sectional side view similar to that shown FIG. 20 but with the instrument shaft and proximal bendable member removed from the handle. FIG. 22 is an exploded perspective view of the embodiment of FIG. 20. FIG. 22A is a fragmentary cross-sectional view taken along line 22A-22A of FIG. 22.

In the embodiment of FIGS. 20-22, the shaft assembly is detachable from the handle. In this way the handle can be reused making the instrument more economically practical. FIG. 21 illustrates where the de-coupling is made between the handle part and the shaft and bendable member part of the instrument. This involves the use of a detachment means. The detachment means 340 includes, on the shaft side, a shaft end adapter 342, and, on the handle side a receiver 348. The detachment means 340 also cooperates with keyed hub 346 in the rotation knob 24. The detachment means 340 is rotatably captured by the receiver 344. A quick disconnect 348 at the receiver 344 captures the ball 350 of the adapter 342 and, at the same time, connects cable 38 to the slider mechanism (not shown) in the handle.

In this embodiment of FIGS. 20-22 it is noted that the main locking mechanism is disclosed as substantially the same as that shown in the embodiment illustrated in FIGS. 12-19. This includes the use of rigid rods 316, split balls 320, support housings 326 and anchor disc 310. This part of the instrument is not now described in detail herein in that it is basically the same structure and operation as previously described in connection with the embodiment shown in FIGS. 12-19.

The adapter 342 may also be considered as including the proximally extending keys 360, extending from the shoulder 364 and the center positioned post 358 that supports the ball 350 and is provided with an annular groove 356 and an end lip 352 with a taper 354. The receiver 344 may be considered as also including a latch gate 368 which is one form of an interlock for holding the two parts of the instrument together. Various types of latching means may be used to secure the two parts of the instrument for use. For example, refer to co-pending applications Ser. No. 11/900,417 filed on Sep. 11, 2007 and Ser. No. 12/006,278 filed on Dec. 31, 2007. Both of these applications are hereby incorporated by reference herein in their entirety. These applications describe various mechanisms for enabling the reuse of the handle part of the instrument, with the shaft portion being disposable or reposable.

To ease the insertion of the shaft with the handle part, there is a taper 354 provided on the lip 352 at the proximal end of the post 358, to aid in lining up with both the rotation knob 24 and the receiver 344. FIG. 21 shows the parts separated while FIG. 20 shows the adapter interlocked with the rotation knob and receiver. When the shoulder 364 of the proximal bendable member is fully engaged with the seat 366 on the rotation knob 24, the gates 368 supported by the receiver 344 are closed (drawn together) to engage the annular groove 356 and lock the shaft in position. FIG. 20 shows the locked position wherein the gates 368 are engaged with the annular groove 356. Concurrently, the cable quick disconnect 348 is engaged with ball 350. The quick disconnect may be of various constructions all including a means for activation of the coupling at the tool actuation cable.

The keys 360 interlock with keyways 362 provided in the keyed hub 346. This interlock links the rotation knob 24 to the proximal bendable member 18 and the instrument shaft 14. In addition, the locking ring has four cams 370 that cooperate with slots 372 in the hub to latch the bezel ring 330 to the hub 202. As can be best seen in FIGS. 22 and 22A, when housings 326 (not shown for simplicity) are seated in mating slots 334 on the hub 202, the cams 370 (shown in dotted outline in FIG. 22) are disposed at the bottom of the lead-ins 374 to the hub slots 372. FIG. 22A in particular shows a fragmentary part of the hub 202 with the slot 372 therein. The slot 372 has a lead-in part 374 that couples to a radial slot portion 376 an past the restriction 378. When the cams 370 are in position aligned adjacent to the lead-in opening of the slot 372, then upon rotation of the rotation knob 24 in the direction of arrow R4 this action forces the cams 370 past restrictions 378 into a position just past the restriction, and thus effectively latching the bezel ring 330 to the hub 202. The length of slot portion 376 allows further rotation of cams 370 to allow cams 332 in slots 328 (see FIG. 18) to fully deploy.

When the shaft part of the instrument is to be detached, then the rotation knob 24 is rotated in the opposite direction past the release position of the cams 332. This action engages cams 370 against restrictions 378. The restrictions can be designed to require considerable force to pass by or frangible portions may be incorporated on the cam portions 370 which will break off to ensure the shaft cannot be reused.

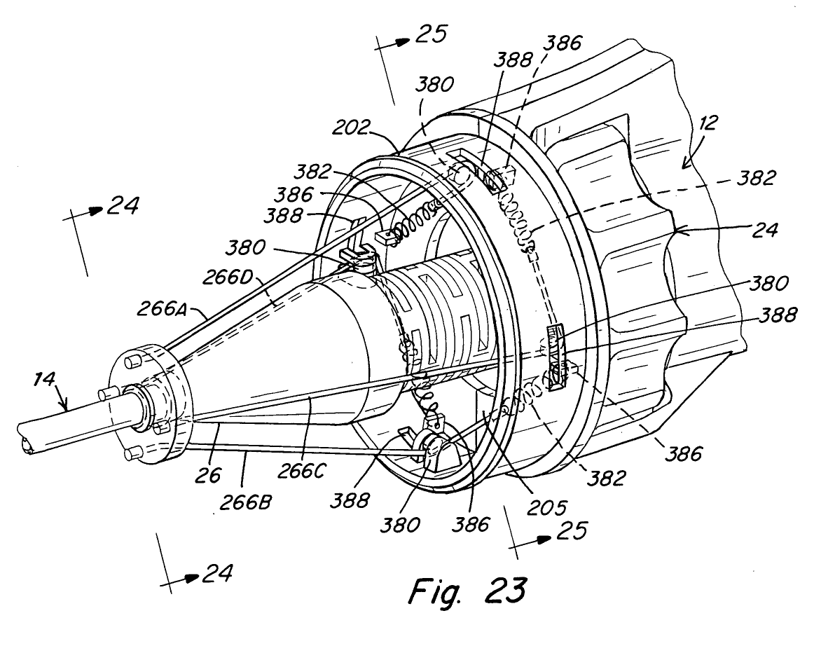

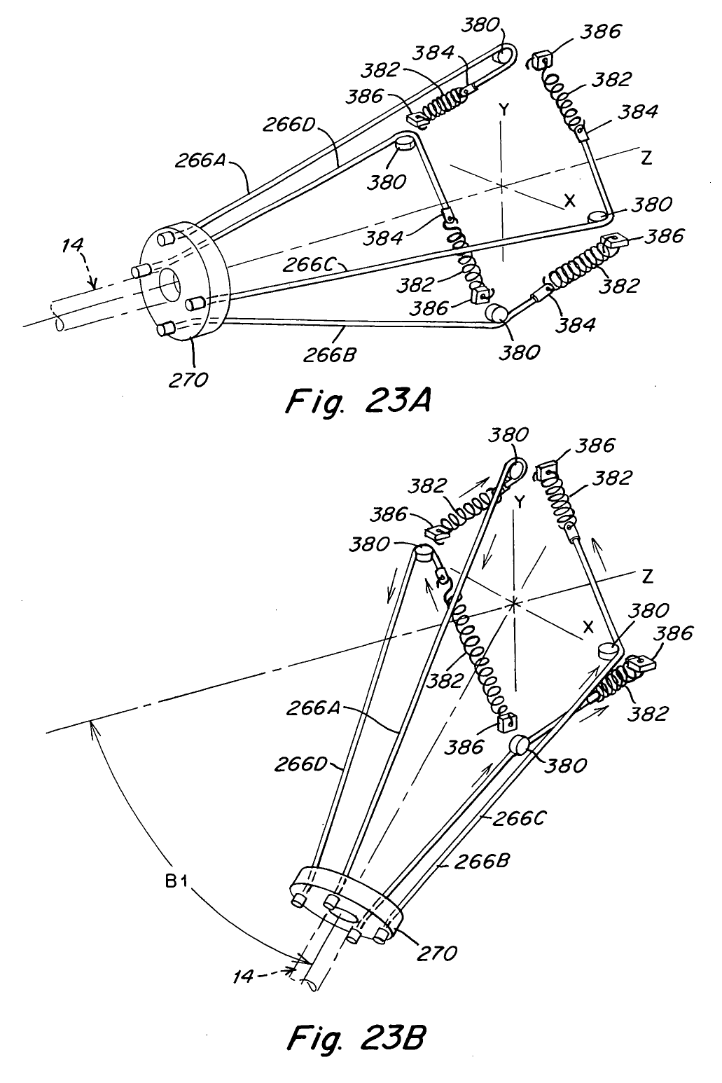

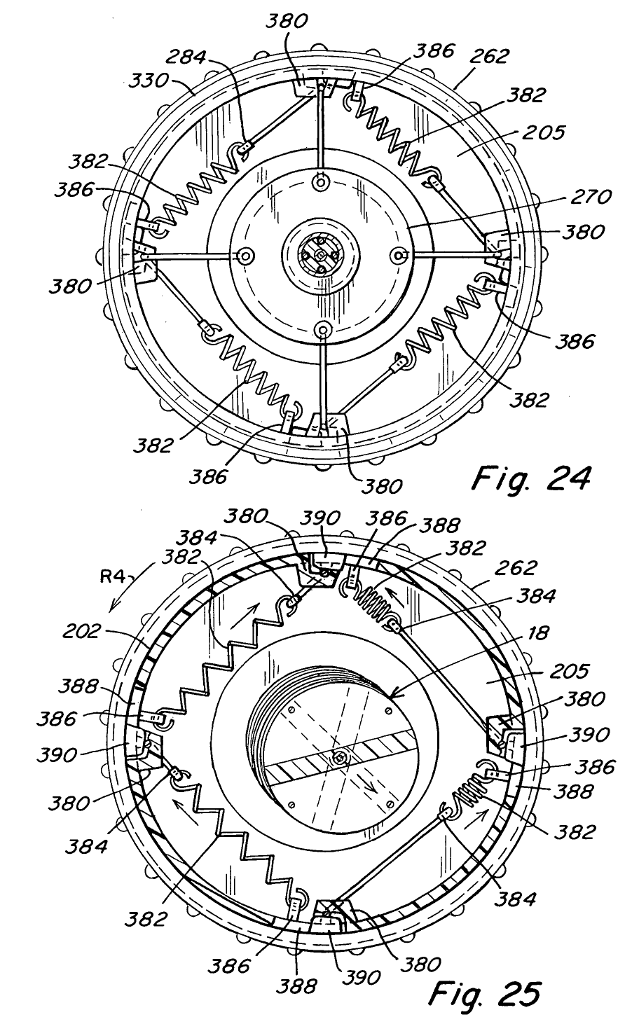

A fifth embodiment of the instrument is shown in FIGS. 23-25. FIG. 23 is a perspective view of this fifth embodiment. FIG. 23A is a schematic perspective view of the cabling mechanism of FIG. 23. FIG. 23B is a schematic perspective view similar to that shown in FIG. 23A but with the instrument rotated in both the “X” and “Y” directions at the same time. FIG. 24 is a cross-sectional view taken along line 24-24 of Fit. 23. FIG. 25 is a cross-sectional view taken along line 25-25 of FIG. 23.

The embodiment of FIGS. 23-25 is similar to the second embodiment illustrated in FIGS. 7-11, but with each cable 266A-266D having its own spring 382 connected at terminal 384 and anchored at 386. In this embodiment of FIGS. 23-25 it is noted a great deal of the structure is substantially the same as that shown in the embodiment illustrated in FIGS. 7-11. This includes the use of cabling 266, locking ring 262, proximal bendable member 18, and in place of pulleys, sheaves 380. Parts of the instrument are not now described in detail herein in that it is basically a similar structure and operation as previously described in connection with the embodiment shown in FIGS. 7-11.

Reference may now be made to FIG. 23, as well as the schematic diagrams shown in FIGS. 23A and 23B, for an illustration of the manner in which the cabling 266 is supported and operated. The cabling has been identified as cable sections 166A, 166B, 166C and 166D. The cabling is actually formed by four separate cables. A spring 382 couples one end of each cable to a fixed anchor 386, as shown in the schematic diagrams. The cabling also passes over the aforementioned sheaves 380. Each of the cables 266A-266D are also fixedly connected at their distal ends to the anchor disc 270. The cables are connected in the X & Y axes so that bending of the shaft in one direction will result in one cable being played out about the sheave 380 and tile opposing cable being reeled in with the spring 382 as a tensioning means.

In the perspective view of FIG. 23 the positioning of the cabling is illustrated. Cables 266A and 266B are connected in the Y axis direction, while cables 266C and 266D are connected in the X axis direction. The springs 382 keep tension on the cables and allow for a small discrepancy between the cable lengths playing in and out as the shaft of the instrument is bent at various angles. As the shaft is angled the cabling simply transitions about the support sheaves. The anchor disk 270 has a bearing surface 274 that seats in raceway 276 on the adapter 26 as seen in FIG. 7, and that allows the shaft and adapter to rotate freely when the rotation knob 24 is rotated. The proximal bendable member 18 and cabling 266 are preferably sheathed in a flexible bellows member. The bellows member may extend around the anchor disk 270 and may be attached at the hub. For the sake of simplicity the sheath is not shown in this embodiment.

Reference is now made to the schematic diagrams of FIGS. 23A and 23B. FIG. 23A shows the position of the cabling when the instrument shaft is in a straight position relative to the handle of the instrument. In other words the angle B1 is zero degrees. FIG. 23B is a schematic perspective view similar to that shown in FIG. 23A but with the instrument rotated through angle B1 which may be in both “X” and the “Y” direction axes. This is a rotation indicated by the angle B1 in FIG. 23B. Refer in FIG. 23B also to the directional arrows that illustrate how the cables are moved over the sheaves to re-position the cables. When the locking ring is then rotated, that pinches the cables at that location locking the position of the instrument.

In FIG. 24 the instrument is shown in an unlocked position wherein the cam 390 is slightly displaced from its associated sheave 380. In FIG. 25 the instrument is shown in a locked position wherein the cam 390 engages its associated sheave 380 with the cable therebetween. This occurs by rotating the locking ring 267 and pinching off the cables at sheaves 380 to lock in the shaft angle.

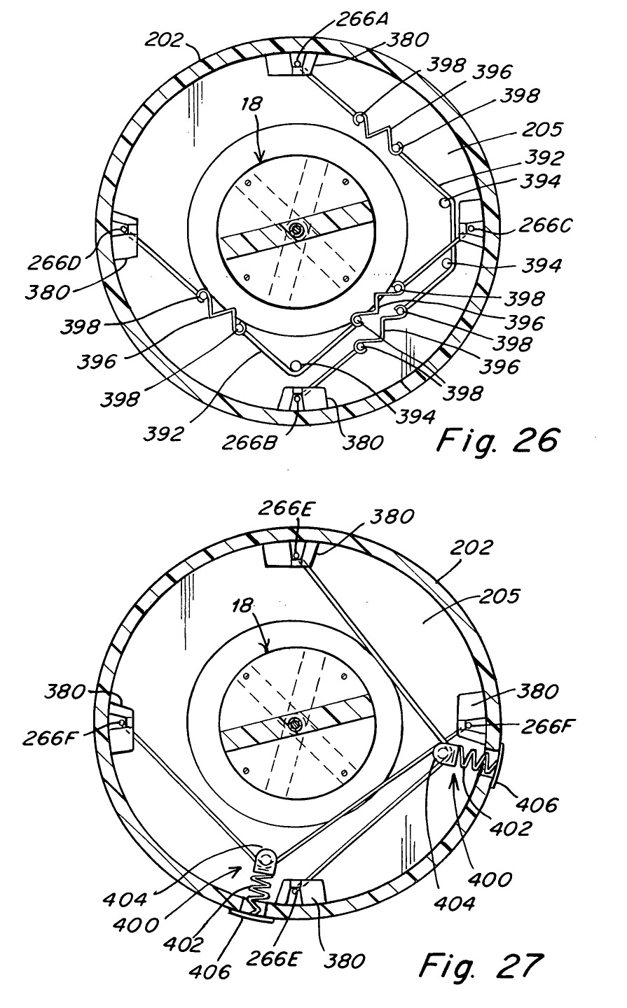

A sixth embodiment of the instrument is shown in the cross-sectional view of FIG. 26. This is a view similar to the cross-sectional view of FIG. 24 but with the cabling disposed in a different pattern. Each of the cables 266A-266D is connected to its own spring 396 by terminals 398. The springs 396 are, in turn, connected to intermediate respective cables 392 that are routed around the proximal bendable member 18 by one or more capstans 394 that are supported from the hub wall 205.

A seventh embodiment of the instrument is shown in the cross-sectional view of FIG. 27. This also is a view similar to the cross-sectional view shown in FIG. 24 but with the cabling disposed in a different pattern. This embodiment includes two cables identified as cables 266 E and 266F. The two cables 266E and 266F loop from the anchor disc 270 around sheaves 380, and are diverted around the proximal bendable member 18. The cables extend about tensioners 400, one for each cable 266E, 266F. The tensioners 400 include pulleys 404 mounted on springs 402 that are attached to the hub 202 by anchors 406.

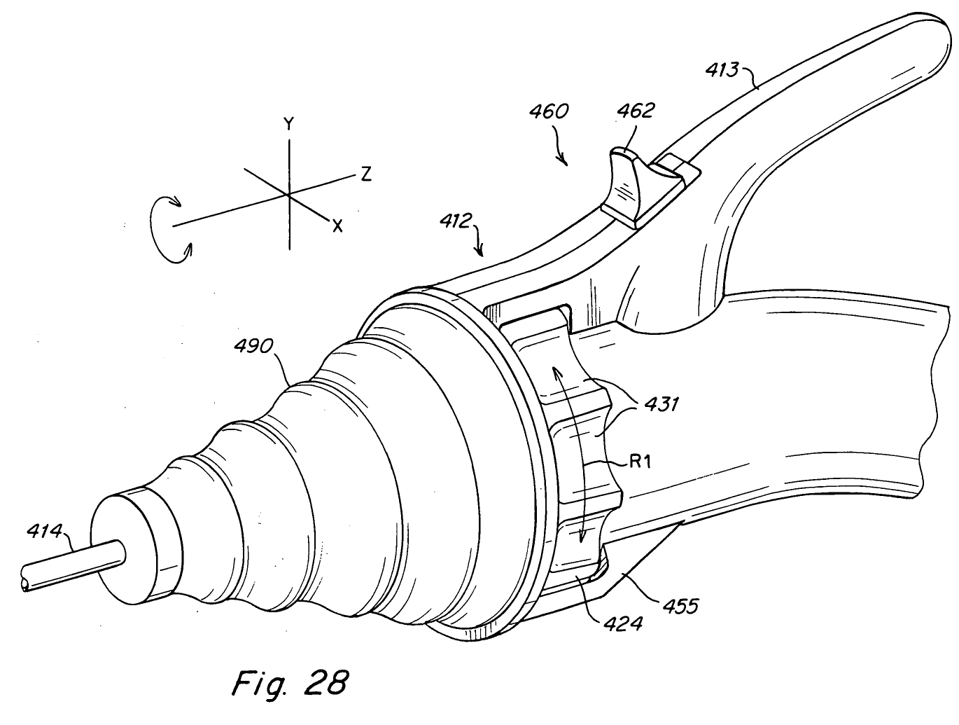

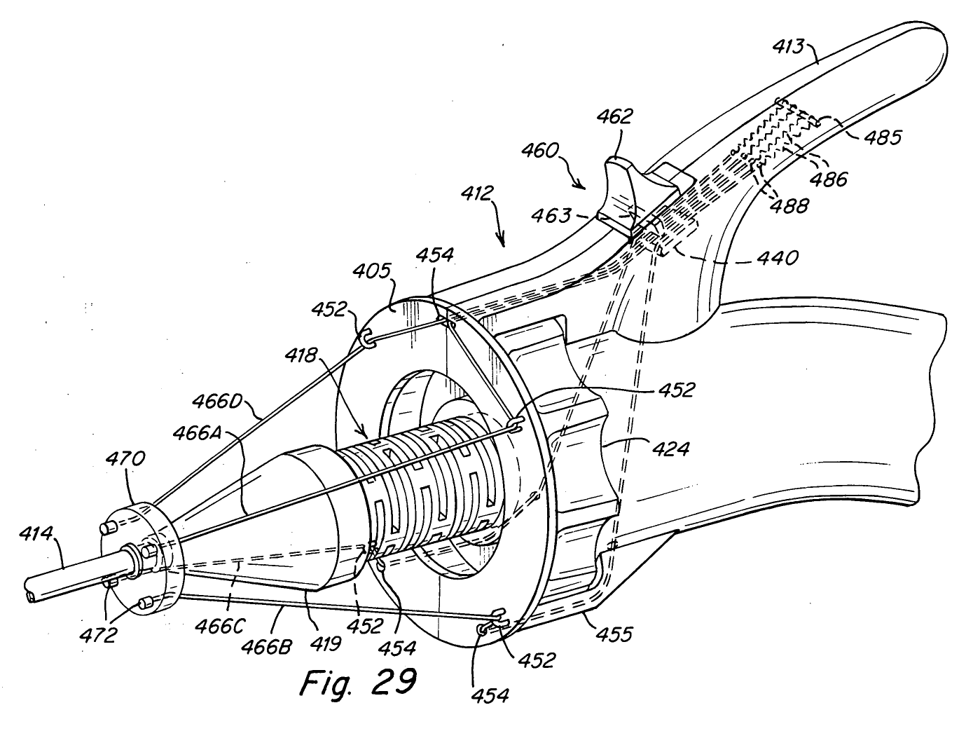

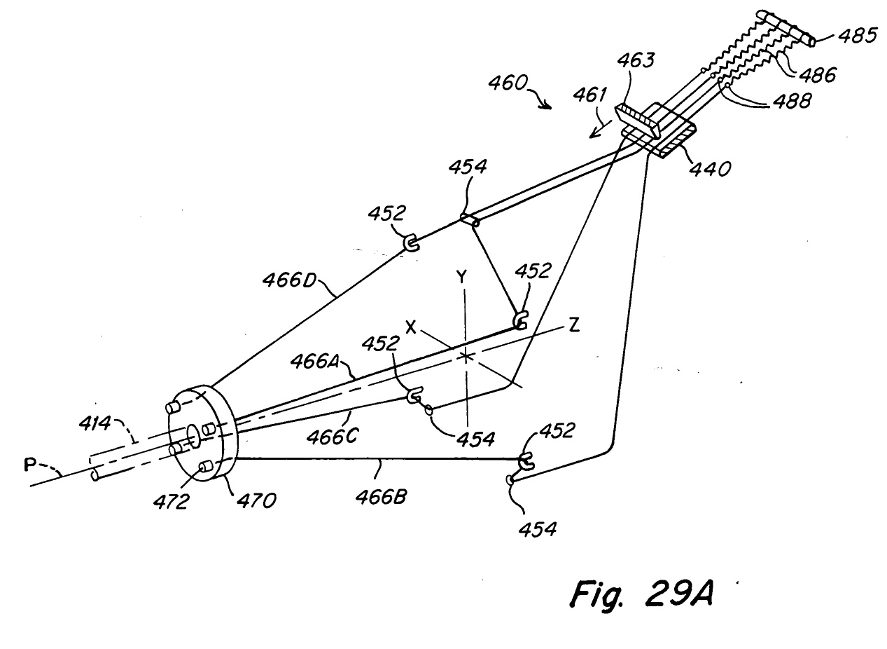

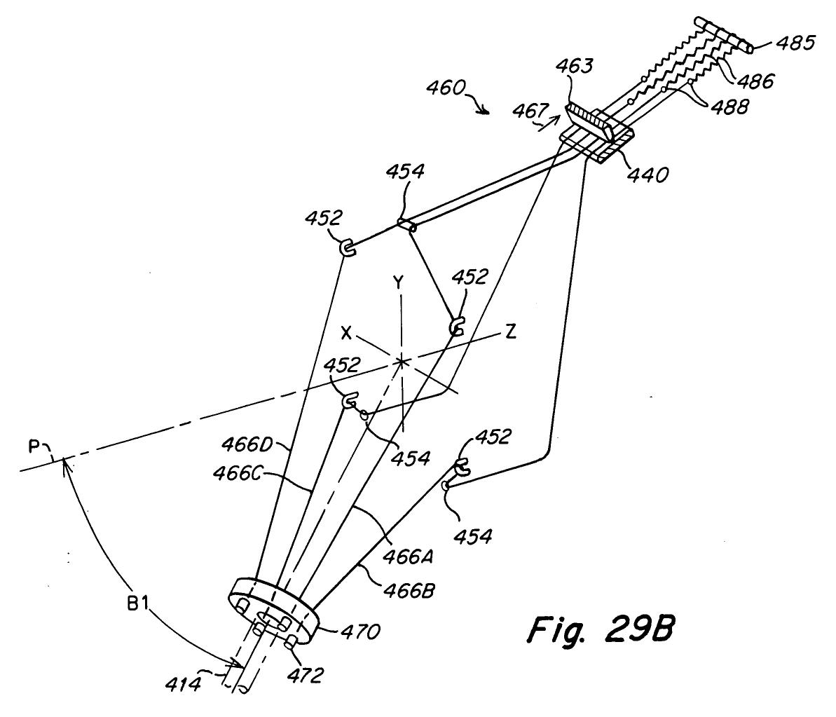

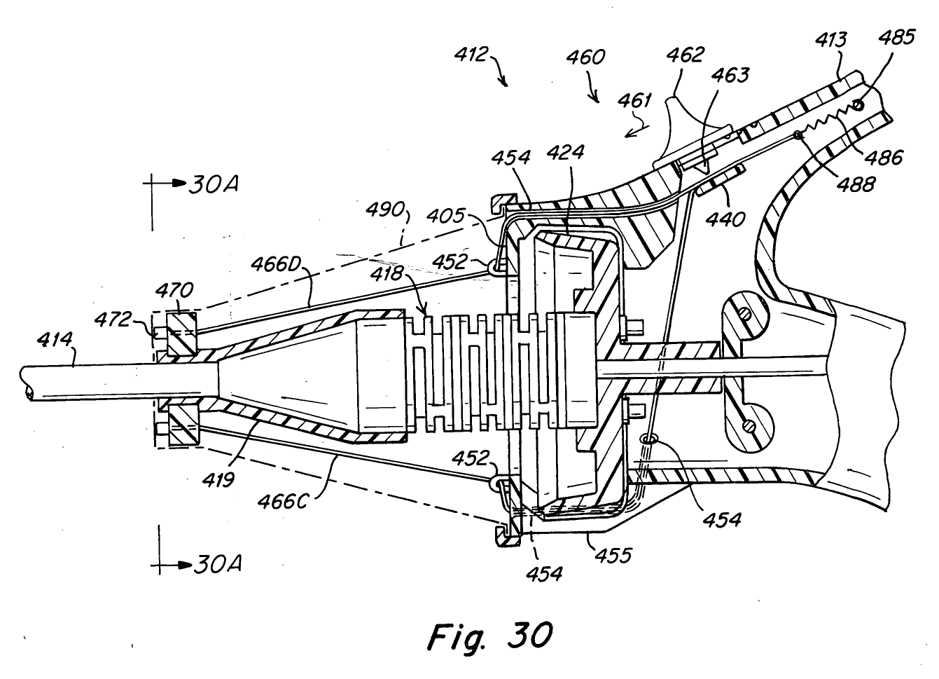

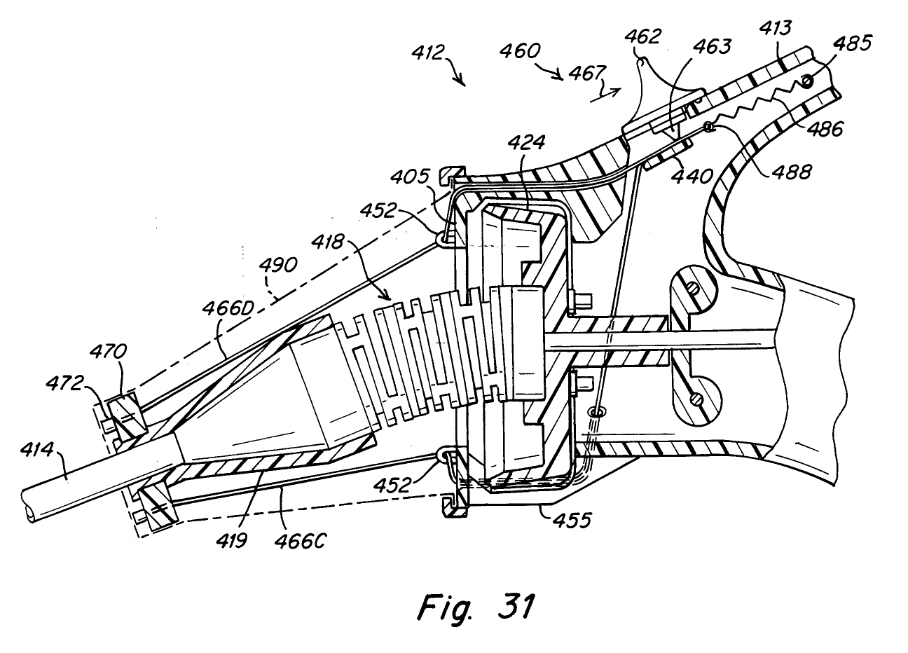

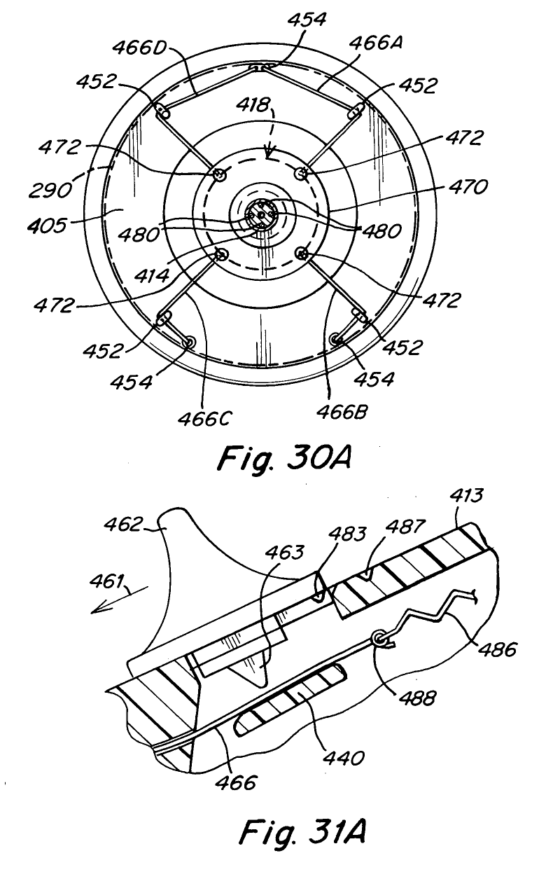

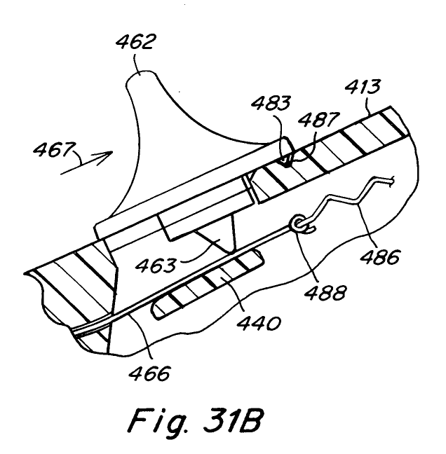

Reference is made to still another embodiment of the present invention illustrated in FIGS. 28-31. FIG. 28 is a fragmentary perspective view of an embodiment of the present invention in which the angle locking member is disposed at the handle, particularly at the horn thereof. FIG. 29 is a fragmentary perspective view like that shown in FIG. 28 with the bellows removed so that the cabling can be seen. FIG. 29A is a schematic perspective view of the cabling mechanism of FIG. 29. FIG. 29B is a schematic perspective view similar to that shown in FIG. 29A but with the instrument rotated in both the “X” and “Y” directions at the same time. FIG. 30 is a cross-sectional side view of the instrument in FIGS. 28 and 29. FIG. 30A is a cross-sectional view taken along line 30A-30A of FIG. 30. FIG. 31 is a cross-sectional side view of the instrument in FIGS. 28 and 29 with the instrument in a bent condition. FIGS. 31A and 31B are fragmentary cross-sectional views taken at the slide button for the respective released and locked positions thereof.

In this particular embodiment the angle locking means or member, instead of being in the form of a locking ring, is embodied as a locking mechanism that is supported more proximally at the handle. It is the particular use of cabling described herein in earlier embodiments that lends itself well to being able to relocate the locking mechanism to any one of a number of different positions on the instrument. This makes it more comfortable in the use of the instrument. In the embodiment of FIGS. 28-31, the entire instrument is not shown as the instrument is basically the same as that described hereinbefore including, in addition to the handle, an instrument shaft, end effector, and distal and proximal bendable members. In this embodiment only the proximal bendable member is disclosed. The control between the proximal and distal bendable members is provided by means of bend control cables that are not specifically illustrated in FIGS. 28 and 29, but that are illustrated by the cables 480 in the cross-sectional view of FIG. 30A.

In the embodiment of FIG. 28 the handle 412 is considered as in the form of a pistol grip and includes an extending horn 413 that facilitates a comfortable interface between the action of the surgeon's hand and the instrument. The shape of the handle allows for a comfortable and substantially one-handed operation of the instrument. The surgeon may grip the handle 412 between his palm and middle finger with the horn 413 nestled in the crook between the thumb and forefinger. This frees up and positions the forefinger and thumb to rotate the rotation knob 424 using the finger indentations 431 that are disposed on the peripheral surface of the rotation knob. In both the locked and unlocked position of the instrument, the rotation knob is capable of controlled rotation to control axial rotation at the tip of the instrument, as shown in FIG. 1 by rotation arrow R3.

The perspective view of FIG. 28 illustrates the angle locking mechanism 460 as comprised of a slide button 462 that is mounted to the top side of the horn 413. However, the angle locking mechanism may also be mounted at other locations on the handle 412. Moreover, instead of a slide button, other forms of actuation means may be provided having respective locked and unlocked or released positions. The fragmentary perspective view of FIG. 28 also illustrates the bellows 490 that extend over the proximal bendable member 418. The instrument shaft 414 is also illustrated. The cross-sectional view of FIG. 30A is taken through the instrument shaft and thus also illustrates the bend control cables 480. The control between the proximal bendable member and the distal bendable member is provided by means of these bend control cables 480. In the illustrated embodiment four such control cables are provided in order to have the all direction bending. However, in other embodiments of the invention fewer or more numbers of bend control cables may be used. The bend control cables extend through the instrument shaft and through the proximal and distal bendable members, basically terminating at the distal end of the distal bendable member and at the proximal end of the proximal bendable member. The bend control cables are preferably constrained along substantially their entire length so as to facilitate both “pushing” and “pulling” action. The cables 480 are also preferably constrained as they pass over the conical cable guide portion 419 of the proximal bendable member 418, and then through the proximal bendable member itself.

Reference is now made to the perspective view of FIG. 29 which discloses further details of this embodiment. The bellows has been removed so as to further illustrate the proximal bendable member 418 and the conical portion 419. The proximal bendable member 418 may be of the type shown in FIG. 2 herein with disks and slots. FIG. 29 also shows the radial wall 405. In this regard refer also to the side cross-sectional view of FIG. 30 that shows the radial wall 405 that is essentially integral with the distal end of the handle 412. The rotation knob 424 is disposed behind the radial wall 405.

In this embodiment of the invention, the proximal bendable member 418 is shown supported between the rotation knob 424 and the instrument shaft 414. This support includes the conical portion 419. The distal end of the conical portion 419 supports the anchor disc 470, as shown in FIGS. 29 and 30. It is the anchor disc 470 that provides the fixed support for the cabling 466. Refer, for example, to FIG. 30A which shows the anchor ring or disc 470 with all four cable ends fixedly attached at 472 to the anchor disc 470.

Reference is now made to FIG. 29 as well as the schematic diagrams illustrated in FIGS. 29A and 29B, for an illustration of the manner in which the cabling 466 is supported and operated. In the drawings the cabling is identified as separate cable sections 466A, 466B, 466C and 466D. Each of these cable sections is fixedly attached to the anchor disc 470 by the crimps 472 at the more distal end of each of the cable sections. At the opposite end of each of the cable sections they are terminated at cable terminal 488 and coupled to respective tension springs 486. The tension springs 486 are also retained by a cross pin 485.

Each of the cables 466 extends from the anchor disc 470 through an eyelet 452. Refer to the perspective view of FIG. 29 which illustrates the eyelets 452 disposed about the periphery and attached to the radial wall 405. Refer also to the cross-sectional view of FIG. 30A which shows the relative positions of the eyelets 452. In place of these eyelets, other means may be provided for guiding the cable section such as a pulley arrangement. Each of the cable sections then extends through spacedly disposed passages 454 in the radial wall 405. As illustrated in FIGS. 29 and 30A, the passage 454 at the top accommodates two cable sections and the two passages at the bottom accommodate respective cable sections. These cable sections extend through struts 455 of the handle 412. Refer also to the cross-sectional view of FIG. 30 which illustrates the cable sections 466 looping through the eyelets 452 and then extending through the handle.

All of the respective cable sections are directed over the ramp 440. In this regard refer to the ramp 440 and schematic diagrams of FIGS. 29A and 29B, as well as to the cross-sectional view of FIG. 30. The ramp 440 may be supported in a number of different ways and is constructed and arranged to be fixed in position and preferably somewhat at a slant relative to the slide button 462, as illustrated in FIGS. 31A and 31B. In this regard, and with reference to FIGS. 30 and 31A, it is noted that the slide button 462 is in its released position as illustrated by the arrow 461 and thus the cam 463 which is carried by the slide button 462 is out of engagement with the ramp 440. FIGS. 30 and 31A illustrate a gap between the cam 463 and the upper ramp surface which is desirable so that the cable sections can move as the proximal bendable member is moved to a bent condition such as is illustrated in the cross-sectional view of FIG. 31. FIGS. 31 and 31B illustrate the slide button 462 moved in the direction of arrow 467 to its locked position. In that position it is noted that the cam 463 has moved in the same direction against the cable sections clamping the cable sections between the cam and the upper surface of the ramp 440. This is thus the locked position of the slide button 462 in which the cable sections are snubbed so as to hold the cable sections at the particular length illustrated. FIGS. 31A and 31B also show an interlock arrangement so that the slide button can be kept in the locked position. This includes the bump 483 on the slide button interacting with the indent 487 in a wall of the horn 413. Other types of interlocks may also be used to hold the slide button in a locked position. Moreover, an arrangement can be used that allows the slide button to be held securely in either locked or release positions.

With further reference to the schematic diagrams of FIGS. 29A and 29B, it is noted that in FIG. 29A the instrument is depicted as in a straight line position with the angle locking means in its released position. In that position, the springs 486 provide a like tension on the cable sections and thus each of the springs is shown as being of substantially the same length. On the other hand, the schematic diagram of FIG. 29B depicts a condition wherein the instrument has been bent at 35 degrees in both the X and Y direction. This is illustrated in FIG. 29B by the fact that the cam has locked the cable sections but the springs, as noted in FIG. 29B are of different lengths. This is representative of the bending action.

The instrument of the present invention provides an improved instrument, particularly from the standpoint of ease of use by the surgeon. The tool actuation lever arrangement permits fine control by the user, particularly with the instrument arrangement that has the recessed gimbal where the finger of the user can be readily engaged with the lever. This arrangement also enables the instrument to be readily adapted for either right-handed or left-handed control by simply rotating the gimbal in its socket between opposite positions. It is also preferred that the recess in the gimbal be formed by a blind hole (with a bottom wall) as this has been found to provide enhanced manual control of the lever positioning.

Another improvement of the instrument of the present invention relates to the ease with which the tool can be controlled including the convenient placement of the rotation member and the convenient placement of the locking arrangement where the users thumb and forefinger can be readily used to control both tip rotation as well as locking. These functions can be performed with a single hand and without requiring the user to move the hand position.

Still another important feature of the present invention relates to providing a medical instrument in which the associated locking mechanism can be made quite compact. This is possible at least in part because the locking feature uses cabling or rigid rods disposed about the proximal bendable member that move with bending action of the instrument and can thus be readily easily pinched or clamped to hold the instrument position. The medical instrument of the present invention also provides substantial flexibility as to the location of the angle locking means, such as at the handle in the last embodiment that is described.

Having now described a limited number of embodiments of the present invention it should now be apparent to one skilled in the art that numerous other embodiments and modifications are contemplated as falling within the scope of the present invention as defined by the appended claims. For example, in another version of the present invention a different form of instrument tip rotation means may be used such as a slide mechanism to control distal rotation about the tool tip axis. Even with such alternate means a locking function may still be associated with the instrument to provide the lock function. The locking means described herein has been illustrated for use with a pistol grip handle, however, this locking means may also be provided on an in-line instrument such as the type illustrated in Ser. No. 11/185,911 filed on Jul. 20, 2005. Also, in the instrument that is described herein the movable members have been illustrated as bendable sections, and more particularly, as unitary bendable sections. However, the movable members may alternatively be of other constructions including, but not limited to, engageable discs, bellows arrangements, a movable ring assembly or ball and socket members. For other forms of bendable members refer to co-pending provisional applications Ser. No. 60/802,885 filed on May 23, 2006 and 60/811,046 filed on Jun. 5, 2006, both of which are hereby incorporated by reference herein in their entirety. Also, in FIGS. 20-22 herein a means is disclosed for detaching the shaft portion of the instrument from the handle portion of the instrument. This detachable feature can also apply to other embodiments disclosed herein, wherein the shaft portion is basically detached from the rotation knob at the instrument handle.

What is claimed is:

1. A surgical instrument comprising:
an instrument shaft having proximal and distal ends;
a tool disposed from the distal end of the instrument shaft;
a control handle coupled from the proximal end of the instrument shaft;
a distal motion member for coupling the distal end of said instrument shaft to said tool;
a proximal motion member for coupling the proximal end of said instrument shaft to said handle;
actuation means extending between said distal and proximal motion members for coupling motion of said proximal motion member to said distal motion member for controlling the positioning of said tool;
and a locking mechanism for fixing the position of the tool at a selected position and having locked and unlocked states;
said locking mechanism including one of a cable array and rod array disposed about said proximal motion member and a locking ring disposed about said proximal motion member and having locked and released positions, and in said locked position engaging said one of a cable array and rod array.

2. The surgical instrument of claim 1 further including a rotation means disposed adjacent the control handle and rotatable relative to the control handle for causing a corresponding rotation of the instrument shaft and tool.

3. The surgical instrument of claim 2 wherein at least said proximal motion member comprises a proximal bendable member, said rotation means comprises a rotation knob that is adapted to rotate the tool about a distal tool roll axis and said rotation knob is disposed between said control handle and proximal bendable member.

4. The surgical instrument of claim 1 wherein said control handle comprises a pistol grip handle having an engagement horn to assist in holding the handle.

5. The surgical instrument of claim 4 wherein said rotation means comprises a rotation knob that is disposed at the distal end of the handle and said horn is disposed proximally of the rotation knob and on the top of the pistol grip handle.

6. The surgical instrument of claim 4 including an actuation lever supported from said pistol grip handle at a pivot point at the proximal end of the handle.

7. The surgical instrument of claim 6 wherein said actuation lever has a free end with a finger loop for receiving a users finger to control the lever.

8. The surgical instrument of claim 1 wherein said one of a cable array and rod array comprises a cable array including a plurality of cable sections that extend about a handle hub, said locking ring disposed about the handle hub and including means for pinching a cable section to hold the position of the instrument shaft.

9. The surgical instrument of claim 8 wherein the cable sections extend about an outer circumferential surface of the handle, the locking ring has an internal cam that pinches the cable section against a rib on the handle hub and including a spring means in each cable section.

10. The surgical instrument of claim 1 wherein said one of a cable array and rod array comprises a cable array including a plurality of cable sections that extend about the proximal motion member, and a plurality of pulleys mounted in a handle hub and supporting respective cable sections.

11. The surgical instrument of claim 10 wherein said handle hub has peripheral slots, said locking ring has peripherally disposed internal cams that pinch the respective cable sections against a pulley and further including spring means in at least one cable section.

12. The surgical instrument of claim 1 wherein said one of a cable array and rod array comprises a rod array including a plurality of separate rods that extend about the proximal motion member, and a plurality of housings that are supported by a handle hub and include a corresponding plurality of split balls that receive respective rods.

13. The surgical instrument of claim 12 including a plurality of peripherally disposed internal cams on said locking ring for engaging said split balls to lock the position.

14. The surgical instrument of claim 1 wherein said locking ring, proximal motion member and instrument shaft are removable from the control handle, and including a quick disconnect means for releasably engaging a tool actuation cable means.

15. The surgical instrument of claim 1 wherein said one of a cable array and rod array comprises a cable array including a plurality of cable sections that extend about the proximal motion member, a plurality of sheaves mounted in a handle hub and supporting respective cable sections, a fixed position anchor disc disposed about the instrument shaft for securing one end of each cable section, a fixed anchor for securing an opposite end of each cable section and a spring disposed in each cable section.

16. The surgical instrument of claim 15 further including capstan means for supporting at least some of said cable sections.

17. In a medical instrument having a proximal control handle and a distal tool that are intercoupled by an elongated instrument shaft that is meant to pass internally of an anatomic body, proximal and distal bendable members that respectively intercouple said proximal control handle and said distal tool with said instrument shaft, cable actuation means disposed between said bendable members, said control handle having proximal and distal ends, an actuation lever for controlling said distal tool, means for pivotally supporting said actuation lever from the proximal end of said handle, and a locking mechanism for fixing the position of the tool at a selected position and having locked and unlocked states, said locking mechanism including one of a cable array and rod array disposed about said proximal bendable member and a locking ring disposed about said proximal bendable member and having locked and released positions, and in said locked position engaging said one of a cable array and rod array.

18. The surgical instrument of claim 17 wherein said one of a cable array and rod array comprises a cable array including a plurality of cable sections that extend about a handle hub, said locking ring disposed about the handle hub and including means for pinching a cable section to hold the position of the instrument shaft.

19. The surgical instrument of claim 18 wherein the cable sections extend about an outer circumferential surface of the handle, the locking ring has an internal cam that pinches the cable section against a rib on the handle hub and including a spring means in each cable section.

20. The surgical instrument of claim 17 wherein said one of a cable array and rod array comprises a cable array including a plurality of cable sections that extend about the proximal motion member, and a plurality of pulleys mounted in a handle hub and supporting respective cable sections.

21. The surgical instrument of claim 20 wherein said handle hub has peripheral slots, said locking ring has peripherally disposed internal cams that pinch the respective cable sections against a pulley and further including spring means in at least one cable section.

22. The surgical instrument of claim 17 wherein said one of a cable array and rod array comprises a rod array including a plurality of separate rods that extend about the proximal motion member, and a plurality of housings that are supported by a handle hub and include a corresponding plurality of split balls that receive respective rods.

23. The surgical instrument of claim 22 including a plurality of peripherally disposed internal cams on said locking ring for engaging said split balls to lock the position.

24. The surgical instrument of claim 17 wherein said locking ring, proximal motion member and instrument shaft are removable from the control handle, and including a quick disconnect means for releasably engaging a tool actuation cable means.

25. The surgical instrument of claim 17 wherein said one of a cable array and rod array comprises a cable array including a plurality of cable sections that extend about the proximal motion member, a plurality of sheaves mounted in a handle hub and supporting respective cable sections, a fixed position anchor disc disposed about the instrument shaft for securing one end of each cable section, a fixed anchor for securing an opposite end of each cable section and a spring disposed in each cable section.

26. The surgical instrument of claim 25 further including capstan means for supporting at least some of said cable sections.

27. A surgical instrument comprising:
an instrument shaft having proximal and distal ends;
a tool disposed from the distal end of the instrument shaft;
a control handle coupled from the proximal end of the instrument shaft;
a distal motion member for coupling the distal end of said instrument shaft to said tool;
a proximal motion member for coupling the proximal end of said instrument shaft to said handle;
actuation means extending between said distal and proximal motion members for coupling motion of said proximal motion member to said distal motion member for controlling the positioning of said tool;
and a locking mechanism for fixing the position of the tool at a selected position and having locked and unlocked states;
said locking mechanism including one of a cable array and rod array having locked and released positions, and in said locked position engaging said one of a cable array and rod array.

28. The surgical instrument of claim 27 wherein the cable sections extend about an outer circumferential surface of the handle, and including a locking ring that has an internal cam that pinches the cable section against a rib on the handle hub and including a spring means in each cable section.

29. The surgical instrument of claim 27 wherein said locking mechanism is mounted at the handle.

30. The surgical instrument of claim 29 wherein the locking mechanism includes a slide button on the handle for capturing the cable sections.
